(19) United States
(12) Patent Application Publication
(10) Pub. No.: US 2012/0202723 A1
(43) Pub. Date: Aug. 9, 2012

(54) POLYOLS AND THEIR USE IN HYDROCARBON LUBRICATING AND DRILLING FLUIDS

(76) Inventors: Kirk J. Abbey, Garner, NC (US); Daniel E. Barber, Fuquay-Varina, NC (US)

(21) Appl. No.: 13/365,776

(22) Filed: Feb. 3, 2012

Related U.S. Application Data
(60) Provisional application No. 61/439,488, filed on Feb. 4, 2011.

(51) Int. Cl. C10M 133/40; C07D 211/22; C09D 7/12; C07C 31/18; C10M 129/14; C09K 8/035; C10M 129/08; C07C 35/14

(52) U.S. Cl. 508/262; 508/583; 546/248; 568/829; 568/852; 507/138; 507/130; 106/287.26; 106/287.2

(57) ABSTRACT

Polyhydroxyl functional compounds that contain an all hydrocarbon backbone, wherein all of the hydroxyl groups are bonded to a primary carbon atom, are prepared through the reaction of an alpha, omega-terminal diol having a total of from about 6 to about 42 carbon atoms terminated with a mono-ol having a total of from about 4 to about 42 carbon atoms. The polyols can be used as additives in hydrocarbon oils, drilling fluids, industrial and automotive lubricating fluids, dispersants, engine lubricants, greases, coatings, adhesives, and also in magnetorheological fluids to improve various properties such as dispersion, wear protection, reduction of friction, high temperature stability, and improved aging.

This application claims the benefit of U.S. Provisional Application No. 61/439,488, filed Feb. 4, 2011, which is herein incorporated by reference.

### FIELD OF THE INVENTION

The present invention relates to poly-hydroxyl functional compounds containing an all hydrocarbon backbone wherein all of the hydroxyl groups are on primary carbon atoms, their manufacture, and their use as dispersants, lubricant and drilling fluid additives when terminated with a mono-ol of greater than or equal to about 6 carbon atoms, and use as a reactive component in adhesives and coatings when prepared without termination or terminated with a mono-ol of less than about 8 carbon atoms. The polyols are also useful as additives in magnetorheological fluids, base oils, and greases.

### BACKGROUND OF THE INVENTION

Guerbet alcohols are produced by a variety of methods such as by reacting two alpha mono alcohols in the presence of heat and a catalyst. An exemplary reaction is illustrated in Scheme A:

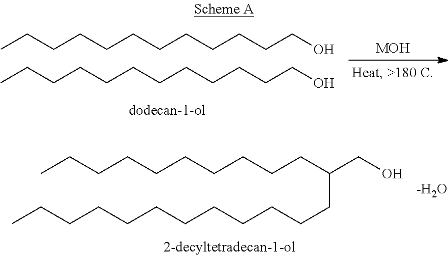

The result is an alcohol with the combined molecular weight of the original alcohols minus the weight of a mole of water with the branching at the beta carbon from the hydroxyl group. The dominant reactions in this Guerbet reaction consists of sequential steps:

1) oxidation of initial alcohol to an aldehyde,

2) condensation of two aldehyde moieties by the Aldol reaction to a beta-hydroxy aldehyde (note one group acts as donor and the other as acceptor),

3) dehydration to form an alpha-beta unsaturated aldehyde, and

4) hydrogenation of both olefin and aldehyde to generate a beta-branched alcohol.

Minor side reactions are known to occur generally under these reaction conditions to generate residual olefinic moieties, carboxylate salts, and sometimes esters by the Tishchenko reaction. Further condensation to give trimers also can occur to some small extent.

U.S. Pat. No. 2,875,241 relates to the Guerbet condensation reaction of glycols which have at least four carbon atoms in a straight chain.

U.S. Pat. No. 3,119,880 relates to the condensation of primary aliphatic alcohols in the presence of lead salt catalysts.

In U.S. Pat. No. 7,049,476, a method for making a polymeric Guerbet alcohol is disclosed for a particular range of ingredients. The primary reaction comprises the reaction of a straight chain diol of 8-12 methylene units, which polymerizes on both ends through the Guerbet reaction, capped with a straight chain mono-ol of 7-22 carbon atoms. The result is a polyol with aliphatic end groups and intermediate methyl-ol groups extending from the main chain. The Guerbet polyols of the '476 patent show utility only in cosmetics and other such personal care applications and are mentioned as useful in metal working and other lubrication applications (Col. 3, lines 30-35).

Publication WO 91/04242 relates to a Guerbet alcohol process that includes the use of certain carbonyl compounds after substantial completion of the reaction to again resume the reaction resulting in increased conversation of the Guerbert alcohol and the use of alcohols, alkoxides and hydrides after completion of the reaction to reduce levels of contaminating compounds.

Thus, there is a desire for improved polyols that can be used in hydrocarbon oils to improve dispersion and wear protection, to reduce friction and viscosity, to improve high temperature stability, and the like. The desired end uses include use in drilling fluids such as drilling muds, automotive and industrial lubricating fluids, and high durability magnetorheological fluids as well as a component in adhesives, coatings, or other polymeric structures.

### SUMMARY OF THE INVENTION

The present invention relates to novel classes of polyols that comprise poly-hydroxy functional aliphatic alcohols wherein the hydroxyl groups are attached to the main chain via methylol moieties or remain as the terminal hydroxyl groups when an uncapped diol is polymerized.

In one embodiment of the present invention, the polyols are prepared through the polymerization of an alpha, omega-terminal diol having a total of from about 4 to 42 carbon atoms and preferably from about 10 to about 36 carbon atoms capped with a mono-ol having a total of from about 4 to about 42 carbon atoms, desirably from about 5 to about 22 carbon atoms, and preferably from about 8 to about 18 carbon atoms. The diols can be straight chain diols or can be branched such as arises from hydrogenating a dimer fatty acid. One commercial source of a fatty dimer diol is Croda's Pripol 2033 (formerly, Uniqema). Another source of fatty dimer diol is Cognis Sovermol 908. The diols and mono-ol can also be cyclic aliphatic or cyclo-hetero-aliphatic provided there is no branching beta to the primary alcohol group, and also aromatic, hetero-aromatic rings where in 1 or >2 carbons separate the hydroxyl group from the aromatic ring. A small portion, 0.5-2.0 equivalent percent, of aromatic terminal functionality is a common but optional feature that arises from the use of a reaction promoting aryl aldehyde.

The novel classes of polyols of the present invention can be used as additives in hydrocarbon or base oils, drilling fluids, industrial and automotive lubricating fluids, greases, dispersants, in an adhesive, or in a coating, and also in magnetorheological fluids to improve various properties such as dispersion, wear protection, reduction of friction, particle settling, and especially high temperature stability and improved aging.

In one aspect of the invention, a polyol composition comprising the reactant of a mono-ol with a diol; wherein said mono-ol, independently, comprises a compound having the formula R—CH₂—CH₂—OH wherein R is a linear alkyl, branched alkyl, cyclic alkyl, or heterocyclic alkyl, or any combination thereof; or a compound having the formula Ar—CH₂—OH wherein Ar is a phenyl, pyridyl, furanyl, m- or p-alkylphenyl, or any other meta or para substituents compatible with the reaction conditions of said mono-ol with said diol; or a compound having the formula Ar′-Q-CH₂—CH₂—OH where Q is —(CR′₂)—ₙ, wherein n is from 1 to about 10, each R′, independently, is H or R as defined above, and wherein Ar′ is phenyl, pyridyl, furanyl, o-, m-, p-alkylphenyl, o-, m-, or p-phenoxyphenyl, or any other ortho, meta, para substituents compatible with the reaction conditions of the mono-ol with said diol; wherein said diol, independently, comprises a compound having the formula R—(CH₂—CH₂—OH)₂ wherein R is a linear alkyl, branched alkyl, cyclic alkyl, or heterocyclic alkyl, or any combination thereof; or a compound having the formula Ar(CH₂—OH)₂ wherein Ar is m-phenyl, m- or m′- or p- or p′-diphenyl ether, or m- or m′ or p- or p′-diphenylmethane, or any other ortho, meta, or para substituents compatible with the reaction conditions of said mono-ol with said diol; or a compound having the formula Ar′(-Q-(CH₂)ₖ—CH₂—OH)₂ where each Q, independently, is —(CR″₂)—ₙ, where each n, independently, is 0 or 1 to about 10 and each R″, independently, can be H or R as defined hereinabove, where each k, independently, is 0 or 1, and wherein Ar′ can be a, o-, m-, or p-alkylphenyl, o-, m-, or p-phenoxyphenyl, or any other ortho, meta, or para substituents compatible with the reaction conditions of said mono-ol with said diol; and wherein said R, Ar, and Ar′ of said different diol formulas can, independently, be the same or different, than said R, Ar, and Ar′ of said mono-ol.

In another aspect of the invention, a process for forming a polyol composition, comprising the steps of reacting a mono-ol with a diol in the presence of a basic catalyst; wherein said mono-ol, independently, comprises a compound having the formula R—CH₂—CH₂—OH wherein R is a linear alkyl, branched alkyl, cyclic alkyl, or heterocyclic alkyl, or any combination thereof; or a compound having the formula Ar—CH₂—OH wherein Ar is a phenyl, pyridyl, furanyl, m- or p-alkylphenyl, or any other meta or para substituents compatible with the reaction conditions of said mono-ol with said diol; and a compound having the formula Ar′-Q-CH₂—CH₂—OH where Q is —(CR′₂)ₙ—, wherein n is from 1 to about 10, each R′, independently, is H or each R as defined above, and wherein Ar′ is phenyl, pyridyl, furanyl, o-, m-, p-alkylphenyl, o-, m-, or p-phenoxyphenyl, or any other ortho, meta, para substituents compatible with the reaction conditions of the mono-ol with said diol; wherein said diol, independently, comprises a compound having the formula R—(CH₂—CH₂—OH)₂ wherein R is a linear alkyl, branched alkyl, cyclic alkyl, or heterocyclic alkyl, or any combination thereof; or a compound having the formula Ar(CH₂—OH)₂ wherein Ar is m-phenyl, m- or m′- or p- or p′-diphenyl ether, or m- or m′ or p- or p′-diphenylmethane, or any other ortho, meta, or para substituents compatible with the reaction conditions; or a compound having the formula Ar′(-Q-(CH₂)ₖ—CH₂—OH)₂ where each Q, independently, is —(CR′₂)ₙ—, where each n, independently, is 0 or 1 to about 10 and each R″, independently, can be H or R′ as defined hereinabove, where each k, independently, is 0 or 1, and wherein Ar′ can be a, o-, m-, or p-alkylphenyl, o-, m-, or p-phenoxyphenyl, or any other ortho, meta, or para substituents compatible with the reaction conditions, or wherein R, Ar, and Ar′ of said diol can, independently, be the same or different than said R, Ar, and Ar′ of said mono-ol.

### BRIEF DESCRIPTION OF THE DRAWINGS

FIG. 1 shows various viscosity increases in magnetorheological fluids after heat aging for 72 hours at 200° C. with respect to fluids containing polyols of the present invention;

FIG. 2 summarizes the Wear Scar diameters of various trials of the present invention;

FIG. 3 summarizes a coefficient of friction of various trials;

FIG. 4 shows that the polyols of the present invention improve the Wear Scar in the presence of ZDDP;

FIG. 5 shows that the coefficient of friction was either unchanged or improved for polyols of the present invention in the presences of ZDDP;

FIGS. 6, 7, and 8 summarize the coefficient of friction, and plastic viscosity of various drilling fluids formulated with polyols of the present invention;

FIG. 9 shows the fluid loss and electrical stability in a drilling fluid with respect to various polyols of the present invention;

FIG. 10 shows that most polyols of the present invention gave an improved coefficient of friction with respect to various base fluids;

FIG. 11 shows pre-aged and aged yield points of drilling fluids containing various polyols of the present invention;

FIG. 12 shows the plastic viscosity of drilling fluids containing various polyols of the present invention;

FIG. 13 shows the HPHT fluid loss of drilling fluids containing the polyols of the present invention;

FIG. 14 shows deceases in electrical stability after heat aging at temperatures of 200° C.;

FIG. 15 shows the lubricity coefficient of drilling fluids containing three different polyol at different concentrations;

FIG. 16 shows the plastic viscosities and yield points of drilling fluids containing the polyols tested in FIG. 15;

FIG. 17 shows the improved filtration resistance in drilling fluids achieved with three polyols and concentrations thereof as set forth in FIG. 15;

FIG. 18 relates to the electrical stability of drilling fluids containing the three noted polyols;

FIG. 19 shows the coefficient of friction values of polyol containing fluids of the present invention with respect to a control; and

FIGS. 20, 21, 22 and 23 show the plastic viscosity and yield points and HTHP filtration and electrical stability of the polyol fluids tested in FIG. 19.

FIG. 24 represents reaction Scheme G1.

### DETAILED DESCRIPTION OF THE INVENTION

According to an embodiment of the present invention, a polyol compound or a polymerized polyol is prepared generally by a Guerbet reaction by reacting one or more mono-ols with one or more alpha-omega terminal diols. The mono-ol comprises a compound having the formula R—CH₂—CH₂—OH where R can be a linear alkyl, branched alkyl such as 3,5,5-trimethylhexanol, cyclic alkyl such as (2-hydroxylethyl)cyclohexane, or heterocyclic alkyl such as N-(6-hydroxyhexyl)piperidine, or any combination thereof, wherein R contains from about 2 to about 40 carbon atoms, desirably from about 3 to about 20 carbon atoms and preferably from about 6 to about 16 carbon atoms. One class of branched mono-ols is the family of “oxo” alcohols such as isononyl alcohol (tradename Exxal 9 from ExxonMobil), isodecyl alcohol (tradename Exxal 10 from ExxonMobil), isotridecanol (tradename Exxal 13 from ExxonMobil), and Safol 23 (tradename Sasol). In lieu of the alkylene mono-ols another embodiment of the present invention comprises an alkylene-aromatic mono-ol having the formula Ar—CH₂—OH wherein Ar comprises from about 4 to about 41 carbon atoms, desirably from about 4 to about 21, and preferably from about 4 to about 17, and can be a phenyl, pyridyl such as 3-hydroxymethylpyridine, furanyl such as furfuryl alcohol (also called 2-(hydroxymethyl)furan), m- or p-alkylphenyl, m- or p-phenoxyphenyl, or any other meta or para substituents compatible with the reaction conditions. Another embodiment of the mono-ol has the formula Ar′-Q-CH₂—CH₂—OH where Q is —(CR′₂)ₙ—, wherein n is 1 to about 10 and preferably from about 1 to about 4 and each R′, independently, can be H or R as defined hereinabove and wherein Ar′ can have from about 4 to about 39, desirably from about 4 to about 19, and preferably from about 4 to about 15 carbon atoms and also can be a phenyl such as in 3-phenylpropanol, 4-phenylbutanol, and the like, pyridyl such as in 4-(3-hydroxypropyl)pyridine, furanyl such as in 3-(4-hydroxybutyl)furan, o-, m-, or p-alkylphenyl, o-, m-, or p-phenoxyphenyl, or any other ortho, meta, or para substituents compatible with the reaction conditions. It is an important aspect of the present invention that desirably the mono-ols have a beta CH₂ carbon atom with respect to the hydroxyl group so that when the mono-ol is reacted with a diol, a beta branch primary alcohol is formed. The actual condensation step in the reaction occurs between the Aldol donor site (beta to the original hydroxyl group and alpha to the aldehydic intermediate) and an Aldol acceptor cite (alpha to the original hydroxyl group and the aldehydic carbon center). Thus, benzyl alcohols can only act as acceptors in the Aldol reaction and never as the donor site. In a still further embodiment of the present invention, the derived polyol is at least partially converted into esters or trimethylsilyl ethers. Another important aspect of the present invention is that not all of the polyols have hydroxyl end groups. That is, at least 1, 2, or 3 hydrocarbon end groups exist.

Suitable diols of the present invention can have the formula R—(CH₂—CH₂—OH)₂ wherein R has from about 2 to about 38 carbon atoms, and desirably from about 6- to about 32 carbon atoms and preferably from about 28 to about 36 carbon atoms, or alternatively, R can have from about 2 to about 10 or from about 6 to about 8 carbon atoms, and R can be a linear alkyl, branched alkyl, cyclic alkyl, or heterocyclic alkyl such as N,N′-bis(10-hydroxydecyl)piperazine, or any combination thereof. Alternatively, the diol can have the formula Ar—(CH₂—OH)₂, wherein Ar can be from about 4 to about 40 carbon atoms and preferably from about 4 to about 34 carbon atoms and can be m-phenyl, m- or m′ or p- or p′-diphenyl ethers such as 3,3′-bis(hydroxymethyl)phenyl ether, or m- or m′- or p- or p′-diphenylmethanes such as 4,4′-bis(hydroxymethyl)diphenyl methane, or 2,5-bis(hydroxymethyl)furan. Another embodiment of the diol has the formula Ar′-Q-(CH₂)ₖ—CH₂—OH where each Q, independently, is —(CR″₂)ₙ—, where each k, independently, is 0 or 1, where each n, independently, is 0 or from about 1 to about 10 and preferably from about 1 to about 4, wherein R″, independently, can be H or R as defined above, and wherein Ar can have from 4 to about 36 or from about 4 to about 32 carbons atoms and can be o-, m- or p-alkylphenyl, o-, m-, or p-phenoxyphenyl, or any other ortho, meta, or para substituents compatible with the reaction conditions. Said R, Ar, and Ar′ of said diols, can, independently, be the same or different than said R, Ar, and Ar′ of said mono-ol.

Catalysts are desirably utilized in preparing polymers derived from an alpha-omega diol with a mono-ol. Basic reagent catalysts are required for promoting the necessary oxidation, condensation, and reduction steps for converting two terminal alcohol groups to a beta-branched alcohol. Examples of basic catalysts include potassium, cesium, or sodium hydroxide or alkoxide or trialkali phosphates or dialkali carbonates, tripotassium phosphate, calcium oxide, potassium bicarbonate, magnesium carbonate, magnesium oxide, sodium metaborate, potassium ethoxide, sodamide, sodium propionate, tricalcium phosphate, potassium butoxide, magnesium trisilicate, potassium acid phosphate (K₂HPO₄), potassium pyrophosphate (K₄P₂O₇), sodium metasilicate, or sodium orthosilicate, or any combination thereof.

Along with the inorganic basic catalyst, a hydrogen transfer catalyst can be utilized such as a transition metal, a transition metal alloy, or a transition metal salt. Although some will be superior to others, examples of hydrogen transfer catalysts include zinc acetate, zinc acetate dehydrate, or other carboxylate salts, zinc molybdenum oxide such as ZnMoO₄, and combinations thereof. It is generally preferred to employ a metal such as nickel, copper, chromium, zinc, tin, silver, cadmium, manganese, cobalt and their oxides and mixed salts. By way of example, but not limited to, the following dehydrogenation catalysts can be used; metallic nickel such as Raney nickel, nickel on kieselguhr, etc., copper chromite; physical mixtures of cobalt and copper; metallic copper; mixtures of a basic oxide, such as calcium oxide, magnesium oxide, or beryllium oxide, and a metal oxide such as copper oxide, with or without smaller percentages of SiO₂, FeO₃ or Al₂O₃; noble metals such as platinum and palladium.

The amount of said basic catalyst is about 1 to 10 parts catalyst and desirably from 3 to 6 parts by weight per 100 parts by weight of the total alcohol. The basic catalyst can all be added initially or incrementally during the reaction. The hydrogen transfer catalyst is generally about 0.01 to about 1.0 parts by weight and desirably from about 0.05 to about 0.5 parts by weight per every 100 parts by weight of the total alcohol.

The molecular weight of the formed polyols is largely determined by the mole ratio of the one or more diols to the one or more mono-ols. Low mole ratios of from about 0.3 to about 1.0, desirably from about 0.4 to about 0.8, and preferably from about 0.5 to about 0.7 give low number average molecular weights, while high ratios such as generally from above 1 to about 10, desirably from about 1.5 to about 5, and preferably from about 2 to about 4 give high number average molecular weights. Low molecular weights are desired in various fluids such as engine lubricant additives, while high molecular weights are desired in drilling fluid additives. Alternatively, the ratios can overlap such as from about 0.5 to about 2.0. In the case where a capping mono-ol is not used, the conversion of the terminal hydroxyl groups is preferably in the 50-95% range, most desirably in the 75-90% range. Very high molecular weights can result in very high viscosities and, because of side reactions, can lead to chemical crosslinking or gelation and thus are avoided.

The reaction is generally carried out at elevated temperatures such as from about 200 to about 270° C. and preferably from about 220 to about 245° C. Suitable conversions are generally obtained after the reaction time of from about 2 to about 24 hours with the shorter time being preferred. Commonly from about 4 to about 8 hours are required. The preparation of the polyols of the present invention is generally carried out in one step.

If insufficient conversion is achieved in a reasonable time, a second addition of basic catalyst can be added to further advance the reaction. If this happens, then an amount of one tenth to three tenths of the originally added catalysts can be used.

In addition to the above catalysts, aldehydes can optionally be used to promote the reaction. Suitable aldehydes are those described in WO 91/04242 including benzaldehyde, tolualdehyde, 4-methylphenylaldehyde, 4-isobutylphenylaldehyde, and the like. Heterocyclic aldehydes such as 3-pyridinecarboxyaldehyde and 2-furaldehyde can also be used. The aldehydes can contain a total from about 5 to 20 carbon atoms. When used, the amount of the aldehyde is generally from about 0.2 to about 5 parts and desirably from about 1.0 to about 3.5 parts by weight per hundred parts alcohol. A portion of these aldehydes become incorporated as terminal moieties in the polyol while the remainder distills out of the reactor. The fraction is largely dependent on the boiling point of the aldehyde.

The reaction mechanisms are complex and not always fully understood. While not being bound by the following reaction schemes, it is thought that the reaction of a diol being capped with a mono-ol is as follows:

For each reaction step in the polymerization, every terminal hydroxyl moiety, except for the case of arylmethanolic types, can be either a donor or acceptor site, but reaction only occurs by a donor reacting with an acceptor. For the arylmethanolic type end groups, this moiety can only act as an acceptor. Thus, the possible structures after only a single step involving cross reaction of a linear, aliphatic mono-ol with a linear, aliphatic diol, where, respectively, a=3-41 carbon atoms and b=6-42 carbon atoms, can have two possible outcomes, Scheme 1. The number of geometric isomers per given chain length increases quadratically as the oligomer length increases with further reaction.

Scheme 1 relates to a reaction wherein a linear, aliphatic mono-ol is reacted with a linear, aliphatic diol.

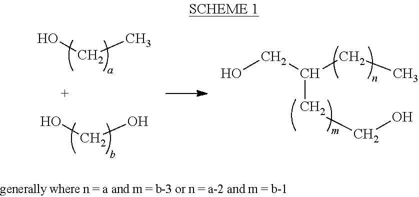

In the above formula, “n” is derived from a mono-ol a that contains from 3 to about 41 and desirably from 4 to about 21 carbon atoms and “m” is derived from the diol b that contains from 6 to 42 and preferably from about 10 to about 36 carbon atoms. As noted above, there are generally two possible outcomes with regard to the length of the chain, n, derived from the alcohol, i.e. n=a or n=a-2 carbon atoms.

Thus, for a final mono-ol terminated oligomer of chain length of six, the number average representation for the reaction of two moles of linear, aliphatic mono-ol and four moles of linear, aliphatic diol at one hundred percent conversion, the structure of Scheme 2 is appropriate where the n's and m's are not fully independent, but are coupled pair-wise between repeating segments.

Scheme 2 relates to an extended reaction between a linear, aliphatic mono-ol and a linear, aliphatic diol.

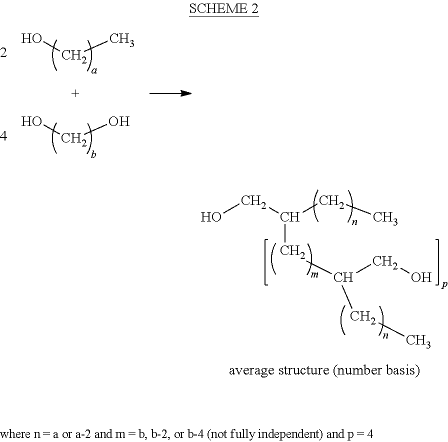

Once again, in the above formulation, “a” contains from 3 to about 41 and desirably from about 4 to about 21 carbon atoms, and “b” contains from 6 to about 42 and desirably from about 10 to about 36 carbon atoms. Upon reaction, independently, n=a or a-2 whereas, m can contain “b” or b-2, or b-4, and p=4. In reality, the reaction is not carried out to complete consumption of the terminal hydroxyl groups even for the mono-ol capped reactions. Some minor amounts of unreacted mono-ol or partially reacted products will still be present. In summary, when the same linear, aliphatic mono-ol and linear, aliphatic diol are used with different initial mole ratio of diol to mono-ol, n and m are always the same as shown above, but p will vary from about 1 to about 20 and can be even higher. On the average, p will be twice the ratio of total moles diol to total moles mono-ol and need not be an integer value.

Thus, it is noted that a side reaction occurs to the extent of about 10% of the hydroxyl groups being converted to carboxylate groups. It is unknown whether this occurs randomly or has some preference to terminal or internal moieties.

Generic structures of the two types of polyol compositions of the present invention are set forth in Scheme 01 and Scheme G2 wherein the mono-ol and the diol is not limited to an aliphatic, aromatic compound.

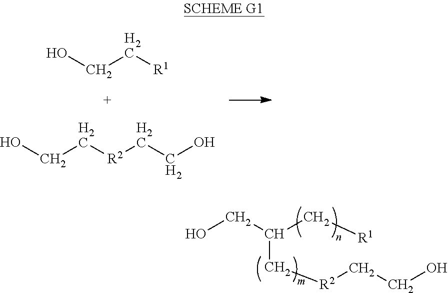

where n is 0 when m is 2 or n is 2 when m is 0.

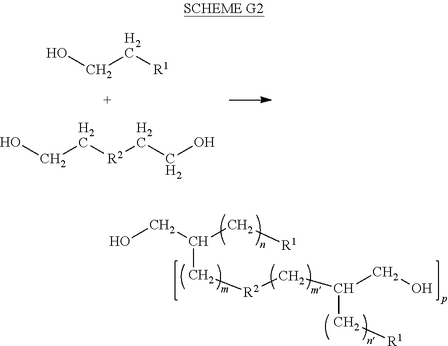

where n is 0 when m is 2, or n is 2 when m is 0;

n′ is 0 when m′ is 2, or n′ is 2 when m′ is 0;

for diol residues not adjacent to terminating mono-ols,

m is 0 when m′ is 2 (of adjacent repeat units), or

m is 2 when m′ is 0 (of adjacent repeat units); and

p is on average two times the molar ratio of diol to mono-ol and need not be integral.

With respect to R¹ of Generic Scheme 1 and Generic Scheme 2 and also with respect to R² of Generic Scheme 1 and Generic Scheme 2, they, independently, can be the same or different compounds.

The Generic R¹ and Generic R² compounds are generally similar or the same as set forth above with regard to Scheme 1 and Scheme 2. With respect to suitable mono-ols, R¹ can be a linear alkyl, branched alkyl such as 3,3,5-trimethylhexanol, cyclic alkyl such as (2-hydroxyethyl)cyclohexane, or heterocyclic alkyl such as N-(6-hydrohexyl)piperidine, wherein R contains from about 1 to about 40 carbon atoms, desirably from about 3 to about 20 carbon atoms and preferably from about 6 to about 16 carbon atoms. One class of branched mono-ols is the family of “oxo” alcohols.

R¹ can also be an aromatic or an alkylene-aromatic mono-ol having the formula Ar—CH₂— wherein Ar comprises from about 4 to about 39 carbon atoms, desirably from about 4 to about 19 carbon atoms, and preferably from about 4 to about 15 carbon atoms, and can be a phenyl, pyridyl such as 3-hydroxymethylpyridine, furanyl such as furfuryl alcohol (also called 2-(hydroxymethyl)furan), m- or p-alkylphenyl, m- or p-phenolxyphenol, or any other meta or para substituents compatible with the reaction conditions. This type of compound forms a special case that will be discussed later.

Yet other compounds of R¹ can have the formula Ar′-Q-CH₂—CH₂—OH where Q is —(CR′₂)ₙ—, where n is from about 1 to about 10 and preferably from about 1 to about 4 and each R″, independently, can be H or R¹ as defined above, and wherein Ar′ can be from 4 to 37 carbon atoms or from 7 to about 17 carbon atoms and can also be a phenyl such as 3-phenylpropanol, 4-phenylbutanol, and the like, pyridyl such as 4-(3-hydroxypropyl)pyridine, furanyl such as 3-(4-hydroxybutyl)furan, o-, m-, or p-alkylphenyl, o-, m-, or p-phenoxyphenyl, or any other ortho, meta, or para substituents compatible with the reaction conditions.

With respect to suitable diols that can be utilized in Scheme 01 and Scheme G2, they are generally similar or identical to the diols utilized in Scheme 1 and Scheme 2 above. That is, R² can have from about 1 to about 38 carbon atoms, and preferably from about 6 to about 32 carbon atoms and R² can be a linear alkylene, branched alkylene, cyclic alkylene, or heterocyclic alkylene such as N,N′-bis(10-hydroxydecyl)piperazine, or any combination thereof. R² can also be an aromatic compound or an alkylene aromatic compound having the formula Ar(CH₂—)₂ wherein R² is as set forth above and wherein Ar is from about 4 to about 36 carbon atoms and preferably from about 4 to about 30 carbon atoms, and can be m-phenyl, m- or m′ or p- or p′-diphenyl ethers such as 3,3′-bis(hydroxymethyl)phenyl ether, or m- or m′- or p- or p′-diphenylmethanes such as 4,4′-bis(hydroxymethyl)diphenyl methane. In another embodiment of the present invention, R² can have the formula Ar′(-Q-(CH₂)ₖ—CH₂)₂ where each Q, independently, is —(CR″₂)ₙ—, where each k, independently, is 0 or 1, where each n, independently, is 0 or from about 1 to about 10 and preferably from about 1 to about 4, wherein each R″, independently, can be H or R² as defined above and wherein Ar′ can have from 4 to 32 and preferably from about 4 to about 28 carbon atoms and can be o-, m-, or p-alkylphenyl, o-, m-, or p-phenoxyphenol, or any other ortho, meta, or para substituents compatible with the reaction conditions.

In the special case when the mono-ol is Ar—CH₂—OH Schemes 3 and 4 set forth the generic reaction thereof.

Special case with ArCH₂OH as mono-ol

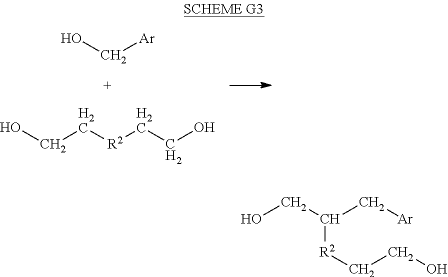

In the above G3 Scheme, Ar comprises from about 4 to about 41 carbon atoms, desirably from about 4 to about 21 carbon atoms, and preferably from about 7 to about 17 carbon atoms, and can be a phenyl, pyridyl such as 3-hydroxymethylpyridine, furanyl such as furfuryl alcohol (also called 2-(hydroxymethyl)furan), m- or p-alkylphenyl, m- or p-phenoxyphenol, or any other meta or para substituents compatible with the reaction conditions.

With respect to Scheme G3, R² of the diols is the same as set forth hereinabove and will not be repeated. The reaction product of the above Scheme G3 mono-ol with the noted diol yields the end products noted on the right hand side of the above and immediately below formulations.

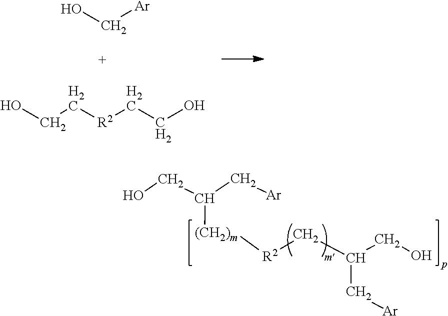

where m=0 and m′=0 when adjacent to a terminal mono-ol

and for diol residues not adjacent to terminating mono-ols

m=0 when m′=2 (of adjacent repeat units)

or m=2 when m′=0 (of adjacent repeat units)

and the average value of all p's equals two times the mole ratio diol to mono-ol

It is noted that in this generic formula, while p is an integer for a particular molecule, the average value of p generally will not be an integer. Note that one can use a mixture of ArCH₂OH and other types of mono-ols to give chains terminating in a statistical mix of the various mono-ols used.

The various reaction conditions with regard to Scheme G1 and Scheme G2 are substantially the same as set forth herein with regard to Scheme 1 and Scheme 2 and are therefore fully incorporated by reference.

Table 1 lists various reacted diols and mono-ols. The first four entries, which correspond to two polyol preparations without any mono-ol capping agent added, were each conducted initially without a hydrogen transfer catalyst. In the first of these, a second addition only of aldehyde led to higher conversion. In the second, similar high conversion was obtained adding the hydrogen transfer catalyst, zinc acetate. (Note: when aromatic aldehydes are used, they become partially incorporated in the polyol, monofunctional aryl aldehydes as chain terminating species. As the aryl aldehydes can only undergo cross-Aldol reactions, once the aliphatic diol is consumed then the polyols are terminated with benzyl alcohol moieties formed by hydrogenation of the aryl aldehyde groups or along with carboxylic acid groups arising from the Cannizarro reaction.)

TABLE 1
Summary of polyols prepared.

| Trial | Diol | Mono-ol | Catalyst | Aldehyde | Conv.^{‡} (%) |
|---|---|---|---|---|---|
| 1a | 1,10-decanediol (C₁₀) | None | None | PhCHO | 65 |
| 1b | ″ | ″ | ″ | 4-OCHPhCHO | 91 |
| 2a | 1,10-decanediol (C₁₀) | None | None | 4-OCHPhCHO | 33 |
| 2b | ″ | ″ | Zn(OAc)₂ | ″ | 89 |
| 3 | 1,10-decanediol (C₁₀) | 1-hexadecanol (C₁₈) | Zn(OAc)₂ | PhCHO | 96 |
| 4 | 1,6-hexanediol (C₆) | 1-heptanol (C₇) | Zn(OAc)₂ | PhCHO | 96 |
| 5 | 1,6-hexanediol (C₆) | 1-hexadecanol (C₁₆) | Zn(OAc)₂ | none | 91 |
| 6 | 1,6-hexanediol (C₆) | 1-hexadecanol (C₁₆) | Zn(OAc)₂ | 4-MePhCHO | 92 |
| 7 | 1,10-decanediol (C₁₀) | 2-MeOPhCH₂OH | Zn(OAc)₂ | 4-MePhCHO | — |
| 8 | 1,10-decanediol (C₁₀) | 4-iBuPhCH₂OH | Zn(OAc)₂ | 4-iBuPhCHO | 88^{†} |
| 9 | 1,10-decanediol (C₁₀) | 1-butanol (C₄) | Zn(OAc)₂ | 4-iBuPhCHO | 81^{#} |
| 10 | 0.297. Pripol blend^{$} | 1-hexadecanol (C₁₆) | Zn(OAc)₂ | 4-MePhCHO | 100 |
| 11 | 0.146 Pripol blend^{$} | 1-hexadecanol (C₁₆) | ZnMoO₄ | 4-MePhCHO | 98 |
| 12 | 0.296 Pripol blend^{$} | 1-hexadecanol (C₁₆) | ZnMoO₄ | 4-MePhCHO | 96 |
| 13 | 0.147 Pripol blend^{$} | 1-hexadecanol (C₁₆) | Zn(OAc)₂ | 4-MePhCHO | 100 |
| 14 | 1,12-dodecanediol (C₁₂) | ½ 3-pyridinemethanol, | Zn(OAc)₂ | 4-MePhCHO | 96 |
| | | ½ 1-hexadecanol | | | |
| 15^{¥} | 1,10-decanediol (C₁₀) | Safol 23 (oxo type) | Zn(OAc)₂ | 4-MePhCHO | 96 |
| 16 | Pripol 2033 | 1-octadecanol (C₁₈) | Zn(OAc)₂ | 4-MePhCHO | 91 |
| 17^{¥} | Pripol 2033 | 1-octadecanol (C₁₈) | Zn(OAc)₂ | 4-MePhCHO | 93 |
| 18^{¥} | Pripol 2033 | 1-octadecanol (C₁₈) | Zn(OAc)₂ | 4-MePhCHO | 98 |
| 19 | 1,12-dodecanediol (C₁₂) | 1-hexadecanol (C₁₆) | Zn(OAc)₂ | 4-MePhCHO | 97 |
| 20^{£} | 1,12-dodecanediol (C₁₂) | 1-hexadecanol (C₁₆) | Zn(OAc)₂ | 4-MePhCHO | 99 |

^{‡}Consumption of initially charged terminal hydroxyl groups
^{†}Based on terminal aliphatic hydroxyl consumption. Unreacted benzyl alcohol ~13.5%.
^{#}Based on terminal aliphatic hydroxyl consumption. No detectable monomers present.
^{$}Mole fraction Pripol 2033 mixed with 1,12-dodecanediol (Pripol 2033 is a diol dimer deriving from a dimer C36 fatty alcohol with a molecular weight of 540, a diol component of more than 94.5% and a hydroxyl value equal to 200-215 mg KOH/g.)
^{¥}Post treated with dimethylsulfate at equal equivalents to KOH used.
^{£}Made with 3x level of KOH to generate a higher acid content.

The reaction temperature of Trials 1a through 20 ranged from about 200 to about 270° C., desirably from about 200 to about 2451, and were from about 330 to about 1350 minutes. The trials of Table 1 were tested for molecular weight, acid value, hydroxyl value, and ash content for selected polyols and the results are set forth in Table 2. The molecular weight is an estimate based on size exclusion chromatography calibrated with polystyrene standards. The theoretical molecular weight is based on the stoichiometry of the diols and mono-ols charged to the reactor assuming complete reaction of the original terminal hydroxyls. The discrepancy is likely embodied in the markedly different hydrodynamic volume of the Guerbet polyols relative to polystyrene.

TABLE 2
Molecular weight, acid value, hydroxyl value, and
ash content for select polyols.

| | | Acid | ROH | Mₙ | | | Ash |
|---|---|---|---|---|---|---|---|
| Trial | Description | (meq/g) | (meq/g) | (theory) | Mₙ | M_{w}/Mₙ | (%) |
| 2b | Uncapped 1,10-decanediol | 0.219 | 6.66 | NA | 4800 | 4.66 | — |
| 3 | 1-Hexadecanol capped 1,10-decanediol | 0.276 | 3.79 | 940 | 2000 | 1.97 | 1.56 |
| 4 | 1-Heptanol capped 1,6-hexanediol | 0.604 | 6.54 | ? | 870 | 2.21 | — |
| 5 | 1-Hexadecanol capped 1,6-hexanediol | 0.280 | 4.29 | 870 | 1020 | 1.30 | — |
| 6 | 1-Hexadecanol capped 1,6-hexanediol | 0.255 | 4.55 | 970 | 860 | 1.62 | — |
| 8 | 4-i-Bu-benzyl alcohol capped | 0.304 | 4.66 | 640 | 1270 | 1.82 | — |
| | 1,10-decanediol | | | | | | |
| 9 | n-Butanol capped 1,10-decanediol | 0.140 | 6.16 | ? | 1840 | 2.28 | — |
| 10 | Design: high Pripol; low diol/monol; | 0.195 | 2.10 | 1190 | 1920 | 2.25 | 1.58 |
| | ZnAc | | | | | | |
| 11 | Design: low Pripol; low diol/monol; | 0.244 | 2.59 | 1150 | 2060 | 2.28 | 1.84 |
| | ZnMo | | | | | | |
| 12 | Design: high Pripol; high diol/monol; | 0.317 | 2.82 | 1670 | 2900 | 2.35 | 1.50 |
| | ZnMo | | | | | | |
| 13 | Design: low Pripol; high diol/monol; | 0.233 | 2.97 | 1590 | 2490 | 3.11 | 1.75 |
| | ZnAc | | | | | | |
| 14 | ½ pyridine terminated | 1.05^{§} | 5.10 | 630 | 1480 | 1.67 | — |
| 15 | “oxo” termination | 0.047^{#} | 3.308 | 540 | 530 | 2.69 | — |
| 16 | 1-Octadecanol capped Pripol 2033 | 0.240 | 2.134 | 2170 | 3010 | 2.04 | — |
| 17 | Repeat of Trial 16 methylated | 0.094 | 1.873 | 2240 | 3310 | 2.53 | — |
| 18 | Repeat of Trial 17 | 0.018 | 1.821 | 2150 | 3280 | 2.13 | — |
| 19 | 1-Hexadecanol capped 1,12- | 0.341 | 3.507 | 920 | 1750 | 1.85 | — |
| | dodecanediol | | | | | | |
| 20 | High acid version of Trial 19 | 1.066 | 4.010 | 920 | 1580 | 1.93 | — |

^{§}Pyridine group also titrated.
^{#}Polyol methylated with dimethylsulfate.

The synthesis of the various polyols from the above trials was as follows:

In the first four trials where no mono-ols were utilized and no hydrogen transfer catalysts, poor results were obtained. In trial 1a the degree of polymerization was only 1.65 and it gave a number average composition of 3.24 hydroxyl groups per molecule. A second aldehyde addition did give a higher conversion. Trial 1b resulted in the mixture being gelled after about two hours and before any significant water condensation was collected. Trial 2a used terephthaldehyde as the only accelerator. The action was much slower than Trial 1a and contained only one-half the conversion after approximately five hours with an estimated degree of polymerization of only about 0.5. Trial 2b which incorporated zinc acetate as a catalyst became viscous and foamy after two hours at temperatures greater than 200° C., but did not gel. The average degree of polymerization was about 6 or 7. It is noted that when monofunctional aromatic aldehydes are used, they often serve as chain terminating species and can be partially incorporated in the polyol. As the aryl aldehydes can only undergo cross-Aldol reactions, once the aliphatic diol is consumed then the polyols are terminated with benzyl alcohol moieties formed by hydrogenation of the aryl aldehyde groups or along with carboxylic acid groups arising from the Cannizarro reaction.

The remaining trials of Table 2, i.e. Trials 3-20, were all reacted with a monofunctional alcohol as a capping agent to limit the degree of polymerization. Trial 3 produced the material originally prepared for screening as a dispersing agent in MR fluids, see below. Trials 10-13 were a fractional factorial design of polyols intended for testing in MR fluids as well. For this design, in addition to the two variables identified in Table 2, i.e. the mole fraction of the two diols and catalyst type, the mole ratio total diol to mono-ol was evaluated at 1.5 and 2.5. Pripol 2033 is a branched diol of inexact structure derived by hydrogenating dimerized fatty acids. The structure is inexact as a mixture of different fatty acids are used that are derived from natural feed stocks and the dimerization process itself generates more than one type of linkage. Scheme A represents a couple of possible structures that might be present. It is to be understood that the reactions are complex and that accordingly different structures exist and thus the present invention is not limited to the structures set forth in Scheme A.

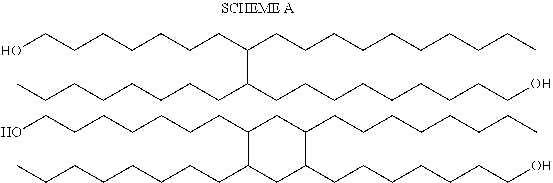

The remaining trials in Table 2 were made to evaluate the range of compositions that could be synthesized. Lower boiling alcohols present a challenge for their use as capping agents because of the very high reaction temperature required for the Guerbet reaction. In trial 4, about half of the initially charged 1-heptanol had to be distilled from the reaction flask before discernible reaction occurred. In trial 9, polymerization was initially attempted under endogenous pressure in a sealed Parr pressure reactor. However, even after ˜5.5 hours at >220° C., less than 10% conversion had occurred. This demonstrated that removal of the water of condensation must occur in order for the reaction to advance.

The same n-butanol composition of Trial 9 was processed further at atmospheric pressure in a reactor by removing butanol until the reaction temperature reached 220° C. Then reaction was continued at 220 t by adding butanol dropwise concurrent with the distillation of butanol and water condensate. After about 220 ml of butanol had been processed, the reaction was allowed to increase in temperature to about 250° C. before removing the heat source. This polyol had considerable terminal hydroxyl content still present, but subsequent gas chromatographic analysis showed that there was no butanol present. The analysis also showed that there was no 2-ethylhexanol, the Guerbet product expected from n-butanol self-condensation. There was also no 1,10-decanediol. Thus, the product polyol consists of oligomers with about 52% of the chain ends terminated with butyl groups and a degree of polymerization of about five.

The use of aromatic mono-ols as capping agents was evaluated in trials 7 and 8. The 2-methoxybenzyl alcohol of trial 7 appeared to have undergone various side reactions including internal transfer of the methyl group possibly as shown in Scheme B. NMR analysis did show some Guerbet reaction had also occurred, but only to a small extent.

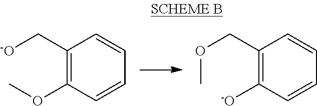

When the 4-isobutylbenzyl alcohol was used instead, trial 8, the reaction went well, but showed the benzyl alcohol moiety was consumed more slowly than the aliphatic hydroxyls of 1,10-decanediol. The initial ratio of aliphatic, terminal hydroxyls to benzylic hydroxyls was 3.0, while in the product mixture, the ratio was 0.9. The residual, unreacted benzyl alcohol was present at about 13.5% of that originally used.

As apparent from Table 2, the ash content of the polyols of the present invention was very low, for example, from about 1.50 to about 1.85. These ash contents are considerably better than the current industry standard zinc dialkyl dithiophosphated (ZDDP) which was approximately 27% ash.

The reactions and preparation of specific polyols of the present invention are as follows.

Uncapped, 1,10-decanediol polyol [Trial 1a]

The 1,10-decanediol (250.14 g; 1.435 moles) and KOH, ˜85% (11.78 g.; ˜0.178 moles) were charged to a custom designed 500 ml resin kettle, equipped with an overhead stirring motor with a bent glass rod as agitator, a thermocouple well, a nitrogen inlet, and a short reflux condenser used as a dephlegmator topped with a Ts 14/20 micro-distillation head. A four-way udder with small round-bottomed flasks attached was connected to the distillation head to receive distillate. The dephlegmator was heated by circulating propylene glycol at a set point of 110° C. The reactor was designed with an outer jacket wherein a Dow Corning silicone fluid with a flash point of greater than 300° C. was placed to act as a heat transfer fluid and to prevent hot spots and possible scorching of the polyol. The filling port for the jacket was equipped with a thermocouple well and a nitrogen blanket linked to the same gas line so as to allow for thermal expansion. An external Glas-Col mantle heated the reactor electrically.

The benzaldehyde (5.90 g; 55.6 mmoles) was added by syringe through the top of the dephlegmator after the reaction temperature reached ˜160° C. The first distillate was collected after the internal temperature reached 204° C. This distillate consisted of two phases. The reaction temperature climbed steadily over about 20 minutes to 240° C. where it was maintained at 241-248° C. for about 1.5 hours before slowly cooling after electrical heating was turned off. A sample labeled Trial 1a was removed for NMR characterization after cooling. The top, organic layer from the distillate was also analyzed, and consisted of benzaldehyde, benzyl alcohol, 1,10-decanediol (mole ratio 0.25:1.00:0.21), and a trace of an unknown.

The reaction was restarted on the next day with terephthaldehyde (5.11 g; 38 mmoles) added. No distillate was collected until the reaction reached 232° C. The temperature rose slowly and steadily to about 256° C. over the next two hours at which point the reactor contents were observed to have gelled.

Uncapped 1,10-decanediol polyol [Trial 2a]

The preceding reaction was repeated with the same equipment with terephthaldehyde being used in the first stage instead of benzaldehyde. The quantities used were 250.47 g (1.437 moles) 1,10-decanediol, 11.72 g (˜0.178 moles) KOH, 85%, and 4.98 g (37.1 mmoles) terephthaldehyde. The first distillate began to collect when the internal temperature reached 213° C. Over the next 1.5 hours, the reaction temperature was maintained in the 225-241° C. range, but only 3.45 g of distillate had collected. After about 1.5 hours of further heating, the distillation head was observed to be blocked by solidifying condensate, most likely 1,10-decanediol. After a brief interruption while the head was cleaned, reaction was allowed to continue for another two hours before turning off the heat. NMR analysis showed very little reaction. The total distillate was 6.44 g while expected was 16.85 g of water.

The next day, the reaction mixture was melted at 94° C. before adding 0.444 g (2.11 mmoles) of zinc acetate dehydrate as a catalyst. Distillate began to collect when the internal temperature reached 237° C. The heat vias turned off after about 1.5 hours during which time >7 g of distillate had collected. The distillation head became blocked several times during this interval and required reaming with a wooden splint to re-open. The reaction was sampled for NMR after cooling, labeled Trial 2a. The reaction product was a stiff, elastic material.

1-Hexadecanol Capped, 1,10-decanediol Polyol [Trial 3]

The ingredients listed below were charged to a custom designed 500 ml resin kettle equipped with an overhead stirring motor with a bent glass rod as agitator, with a custom designed dephlegmator, thermocouple well, and nitrogen inlet. The dephlegmator was connected further to a graduated receiver, and that to a condenser equipped with a gas outlet adapter connected to an oil bubbler. The dephlegmator was operated at 110° C.

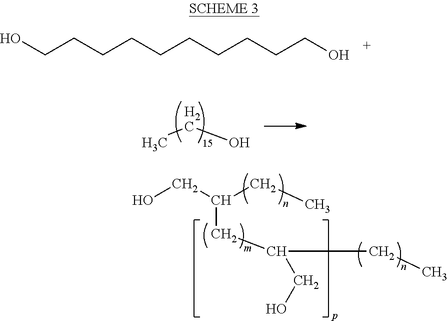

wherein n is 15 or 13, m is 10, or 8, or 6, and p is 1 to 20 with an average of about 3.

The following compounds were utilized in Scheme 3

Decane-1,10-diol, 200.8 g (1.148 moles);

Hexadecan-1-ol, 185.46 g (0.765 moles);

Potassium Hydroxide, 85+%, 11.90 g (˜0.18 moles);

Zinc acetate dehydrate, 98%, 0.147 g (0.0007 moles);

Benzaldehyde, 1.146 g (0.011 moles);

Zinc acetate dehydrate, 98%, 0.476 g (0.0023 moles); and

Benzaldehyde, 5.5 ml (0.054 moles)

The first five materials were heated at 70 volts under a nitrogen atmosphere until the alcohols had melted. The agitation was started and heating continued. Material began to collect in the receiver when the reaction temperature reached 200° C. Fifteen minutes later, the internal temperature had reached 229° C. and the silicone fluid was 268° C. The voltage was reduced to 60 to slow the heating. Eight minutes later, the voltage was further reduced to 55 as the silicone oil had reached 273° C. and the internal temperature was 242° C. The total distillate was only approximately three milliliters.

After a further 1.75 hours only a total of slightly more than 10 milliliters of distillate had collected. A small portion of this was an upper organic layer. The heat was shut off and a sample was removed from the reactor for NMR analysis. It showed very little reaction had occurred.

A further addition of zinc acetate and benzaldehyde were than added, items 6 and 7 above, and heating was resumed at 70 volts. Within ten minutes, distillation had increased to a steady rate. With the reaction temperature slowly rising from 213 to 250° C. over the next twenty minutes, a further 15 milliliters of distillate had collected. Over a further 66 minutes during which time the internal temperature slowly claimed to 270° C., a further three milliliters were collected. The power to the mantle was turned off and the reaction was allowed to slowly cool over the next hour. An additional two milliliters of distillate collected, the internal temperature had cooled to 192° C., and a sample was pulled for NMR analysis. Of the total 30 ml of distillate, about two milliliters of a top layer of organic material were present. When the contents had cooled to 148° C., the reactor was opened and the contents pouted into an amber, tarred, wide-mouthed bottle. The recovered product weighed 349.5 g. This represents 93% recovery of the charged ingredients corrected for the distillate removed.

NMR analysis in deuterochloroform showed greater than 9% of the hydroxyl content being of the primary alcohols of the product. The ratio of terminal methyl signals, from hexadecanol, to the internal hydroxyls at full conversion should have been 6:8. Experimentally, the ratio was found to be 6.00:6.13. The residual unreacted end groups would contribute another 0.31. A majority of these terminal hydroxyls are expected to derive from the diol. Therefore, some loss of hydroxyl functionality to side products, as is known from the synthesis of mono-ols, is evident. These by-product functionalities can be acids, aldehydes, and or olefins.

After fully cooling, the polyol was a hazy, very viscous fluid. The materials had the consistency of soft taffy. The haze may arise from zinc salts or silicates.

Synthesis of 1-Heptanol-Capped, 1,6-Hexanediol-based Guerbet Polyol [Trial 4]

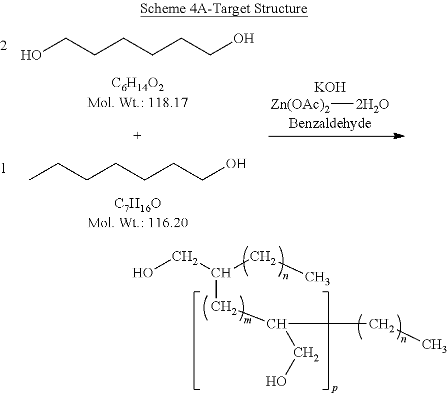

wherein n is 6 or 4, m is 6, or 4, or 2, and p has an average value of 3.

Since the reaction desirably is not carried out to completion, there will be some unreacted components as well as compounds that do not have the above formulation.

A 500 ml, jacketed reactor was charged with 204.69 g (1.732 moles) of 1,6-hexanediol and 100.14 g (0.8618 moles) 1-heptanol. The reaction flask was flushed with nitrogen and heated to melt the alcohols. Overhead stirring was provided with a bent glass rod as agitator. The temperature was monitored by thermocouple inserted in a glass well. The following were added after the reaction mixture was melted and at ˜95° C.: 7.593 g (71.55 mmoles) benzaldehyde, 0.509 g (2.319 mmoles) zinc acetate dihydrate, and 11.83 g (10.05 g active, 0.1792 moles) 85% potassium hydroxide pellets. The reactor was equipped with a trap and condenser.

The reaction was heated for about 50 minutes at which time a steady collection was occurring in the trap. The internal temperature was about 200° C. Over the next 50 minutes, the temperature only rose slowly to about 206° C. The heat was then shut off. After cooling for 25 minutes to an internal temperature of 152° C., a sample was removed from the reactor as well as the top layer in the trap for NMR analysis. The trap contained about 23 ml of top layer and ˜7 ml of bottom layer (presumably water). Only a trace of desired reaction was indicated. The top layer from the trap contained mainly 1-heptanol with traces of benzyl alcohol and benzaldehyde.

The next day, the overhead was changed to use a Dean-Stark trap with bottom stopcock so that heptanol could be removed. An additional 3.589 g (33.82 mmoles) benzaldehyde was charged to the reactor and heating was resumed. Distillate of 1-heptanol and water were collected over about one hour while the reaction temperature steadily climbed from about 200° C. to about 231° C. A total of 67.73 g of distillate were removed. No further 1-heptanol was removed, but water steadily collected as a lower layer in the trap over the next 1.5 hours. About 16 ml of water collected during this time. The heat was turned off and the reactor allowed to cool. When the internal temperature dropped to 184° C., a sample was then removed for NMR analysis. The spectrum was collected in perdeutero-methanol.

The spectrum showed that very little terminal hydroxyls remain. Traces of aliphatic aldehyde, 8.55 ppm, benzoate, 7.95 ppm, benzylic end groups, 2.5-2.7 ppm as two doublet of doublets, and benzyl alcohol, 4.6 ppm, can be seen as well. (The benzylic end groups hydrogens are distinct because of the adjacent chiral center.)

Based on the integration of the NMR spectrum, the approximate degree of polymerization was found to be about eight. The average product polyol has 58% heptyl end groups and ˜42% terminal hydroxyl groups.

Synthesis of 1-Hexadecanol-Capped, 1,6-Hexanediol-Based Guerbet Polyol [Trial 5]

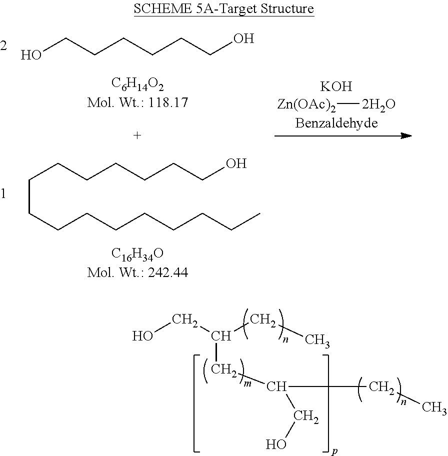

n=14 or 16 independently;
m=6, 4, or 2 independently per repeat unit (4 on average)
(where the independence is conditional);
p=4 on average

A 500 ml, jacketed reactor was charged with 200.28 g (1.695 moles) of 1,6-hexanediol, 205.17 g (0.8463 moles) 1-hexadecanol, and 56.90 g mesitylene. The reaction flask was flushed with nitrogen and heated to melt the alcohols. Overhead stirring was provided with a bent glass rod as agitator. The temperature was monitored by thermocouple inserted in a glass well. The following were added after the reaction mixture was melted and at ˜59° C.: 0.600 g (2.733 mmdes) zinc acetate dihydrate, and 11.47 g (9.750 g active, 0.1738 moles) 85% potassium hydroxide pellets. The reactor was equipped with a Dean-Stark trap with bottom stopcock and condenser.

The reaction was heated for about 55 minutes at which time a steady collection was occurring in the trap. The internal temperature was about 202° C. Over the next ˜15 minutes, the temperature rose slowly to about 222° C. as the trap filled with mesitylene upper layer and ˜4 ml lower aqueous layer. Over the next 45 minutes, the lower layer was occasionally drained. The temperature oscillated between ˜220 and ˜230 as water displaced mesitylene back into the reactor. About 17.1 g of water was collected during this time.

Over the next 16 minutes, all of the distillate was drained while the internal reaction temperature climbed to 235° C. The heat was turned off, but the temperature continued to climb for the next 15 minutes to a peak of ˜251° C. while more mesitylene and water was collected. After another ˜15 minutes, the internal temperature dropped back to ˜240° C. at which point no further distillate was collected. When the internal temperature dropped to ˜176° C., a sample was then removed for NMR analysis, labeled 9550-55B. A proton spectrum was collected in perdeutero-methanol.

The spectrum showed about one-third terminal hydroxyls remain. Traces of aliphatic aldehyde, 8.55 ppm and olefinic hydrogens, 4.6-5.6 ppm can be seen as well. No aryl aldehyde was used in this synthesis.

Based on the integration of the spectrum, the approximate degree of polymerization is found to be less than the target of three. The total hydroxyl content is less than expected at this degree of polymerization applying about ½ of the expected hydroxyls have oxidized to the carboxylic acid salt derivative, Scheme 5B. That is, a small portion of the hydroxyls have been oxidized to a carboxylic acid group and the location of the attachment thereof to the compound set forth in Scheme 5B is generally unknown. Of the total original hydroxyl end groups, the small portion oxidized to a carboxyl group is approximately 10%, plus or minus 2%, or 3%, or 4%.

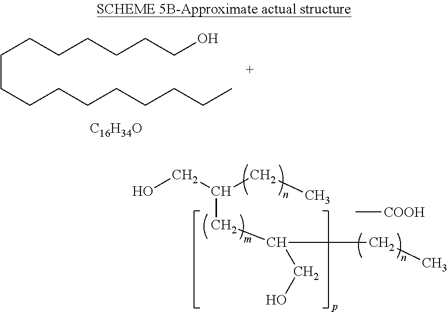

(random intermixed hydroxyls and carboxylates, not blocky as drawn)
n=15 or 13 independently (13 on average);
m=6, 4, or 2 independently per repeat unit (4 on average);
p=<2 on average

wherein n is derived from an alcohol having the formula HO—(CH₂)ₐ—CH₃ where a is from 3 to about 41 carbon atoms;

wherein m is derived from a diol having the formula HO—(CH₂)_{b}—OH where b is from about 6 to about 42 carbon atoms;

wherein a independently=a or a-2 and m=6, or 4, or 2, and p is generally 1 to about 10.

Design: 1-hexadecanol capped, 1,10-decanediol-co-Pripol 2033 [Trials 10-13]

Four resins were prepared in a process similar to Trial 3 above except that a Dean-Stark trap was used to collect water and mesitylene that was used to azeotropically assist water removal. The ingredients and amounts are summarized in Table 3. (An error in Pripol 2033 (Uniqema) molecular weight used in setting up the design was only uncovered subsequently, resulting in the ratios being different from those intended.) The dodecanediol was from Invista (C12™ LD, 98+%) and the hexadecanol was from P&G Chemicals (cetyl alcohol; CO-1695, 95+% with <5% C₁₄ and C₁₈ alcohol).

TABLE 3
Fractional factorial design using Pripol 2033.

| Ingredients | MW | Trial 10 | Moles | Trial 11 | Moles | Trial 12 | Moles | Trial 13 | Moles |
|---|---|---|---|---|---|---|---|---|---|
| Pripol 2033 | 540.58 | 103.30 | 0.191 | 59.20 | 0.110 | 123.20 | 0.228 | 71.90 | 0.133 |
| 1,12-Dodecanediol | 202.34 | 91.73 | 0.453 | 129.66 | 0.641 | 109.56 | 0.541 | 118.01 | 0.583 |
| 1-Hexadecanol | 242.45 | 133.34 | 0.550 | 137.94 | 0.569 | 95.47 | 0.394 | 99.93 | 0.412 |
| p-Tolualdehyde | 120.15 | 4.92 | 0.0410 | 5.03 | 0.419 | 5.32 | 0.0443 | 5.04 | 0.0420 |
| KOH, 85% | 56.11 | 9.84 | 0.149 | 9.98 | 0.151 | 9.86 | 0.149 | 9.82 | 0.149 |
| Zn(OAc)₂•2H₂O | 219.51 | 0.490 | 0.0022 | — | | — | | 0.491 | 0.0022 |
| ZnMoO₄ | 225.33 | — | | 0.564 | 0.0025 | 0.571 | 0.0025 | — | |
| Mesitylene | 120.19 | 46.95 | 0.391 | 47.91 | 0.399 | 48.09 | 0.400 | 45.37 | 0.377 |

| | | | | |
|---|---|---|---|---|
| Mole % Pripol of diols | 29.7 | 14.6 | 29.6 | 14.7 |
| Mole ratio diol/mono-ol | 1.17 | 1.32 | 1.95 | 2.20 |

| | | | | |
|---|---|---|---|---|
| Theoretical molecular weight | 1194 | 1149 | 1667 | 1593 |

In all reactions, some water collected at a reaction temperature of less than 220° C. as the azeotrope with mesitylene carried initial water from the reactor. In all cases except Trial 11, the reaction mixture foamed into the trap and condenser about half way through the reaction. This type of problem was not found in any other reaction and may reflect a specific problem with using mesitylene. Generally, faster agitation rates were instituted to break the foam and the remainder of the reaction was completed successfully.

Synthesis of N-(6-hydroxyhexyl)piperidine

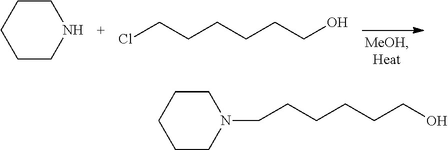

A 100 ml, three-necked, round-bottomed flask equipped with an overhead stirrer, reflux condenser, nitrogen blanket, and thermocouple well was charged with 15.067 g (0.175 moles) piperidine, 25.480 g (0.179 moles) 6-chloro-1-hexanol, and about 10 ml of methanol. The mixture was refluxed at 60-80° C. for 5 hours. Sodium hydroxide (2.526 g, 0.063 moles) were then added and heating continued for about 8 hours. Proton nuclear magnetic resonance (NMR) showed essentially complete consumption of the chlorohexanol.

The reaction mixture was treated with an additional 2.322 g (0.058 moles) of sodium hydroxide and then filtered. The salt was rinsed with four portions of diethyl ether. The ether was removed from the combined organic portion using a rotary evaporator under aspirator pressure. The residue was then distilled using a kugelrohr apparatus at 0.8-0.9 torr to yield 20.58 g of product.

Synthesis of a Polyol with Piperidyl End Groups

To a 1-L reaction kettle, 60.02 g (0.3238 moles) N-(6-hydroxyhexyl)piperidine, 349.66 g (0.6476 moles) Pripol 2033, 10.17 g (0.151 moles active) 85% KOH, 0.6996 g (3.26 millimoles) zinc acetate dihydrate, and 5.06 g (31.2 millimoles) 4-isobutylbenzaldehyde were charged. The reactor was equipped with an overhead stirrer, nitrogen inlet, thermocouple well, dephlegmator, Dean-Stark trap, condenser, and nitrogen outlet. Heating was provided by an electric mantle. After heating at about 225° C. for 22.5 hours, the conversion was about 92%. A further portion of KOH (4.3608 g, 0.0661 moles), zinc acetate dihydrate (0.1360 g, 0.63 millimoles), and isobutylbenzaldehyde (2.08 g, 12.8 moles) was added and heating continued for an additional 4 hours. The final product showed conversion greater than 98% of the initial terminal hydroxyl groups based on proton NMR. Analytical characterization showed the product had a number average molecular weight of about 1,860 and a polydispersity of 2.50. The hydroxyl content was 2.80 meq/g.

Hydrocarbon Oils

The polyols of the present invention can be used as additives in various hydrocarbon oils to improve, as noted above, various properties such as wear protection, dispersion, reduced friction as well as viscosity, and improved high temperature stability. Various hydrocarbon oils include base oils, magnetorheological fluids, drilling fluids, as well as industrial and/or automotive lubricating fluids.

The various polyols of the present invention can be utilized as an additive in base oils. Base oils can be the same as set forth with respect to the magnetorheological fluids set forth below and hereby fully incorporated by reference, or they can be generally defined as natural fatty oils, mineral oils, polyphenylethers, dibasic acid esters, neopentylpolyol esters, phosphate esters, synthetic cycloparaffins and synthetic paraffins, synthetic unsaturated hydrocarbon oils, monobasic acid esters, glycol esters and ethers, silicate esters, silicone oils, silicone copolymers, synthetic hydrocarbons, poly-alpha-olefins derived from oligomerizing terminal alkenes such as 1-butene, 1-hexene, and the like, poly-alkylene-glycols such as oligomeric poly(propylene oxide), poly(butylenes oxide), and various alkylene oxide copolymers, naphthenic oils, diesel oils, and mixtures or blends thereof.

Magnetorheological fluids are known to the literature and to the art and generally comprise magnetic field responsive fluids containing a field polarizable particle component and a liquid carrier component. Magnetorheological fluids are useful in devices or systems for controlling vibration and/or noise. Magnetorheological fluids have been proposed for controlling damping in various devices, such as dampers, shock absorbers, and elastomeric mounts. They have also been proposed for use in controlling pressure and/or torque in brakes, clutches, and valves. Magnetorheological fluids are considered superior to electrorheological fluids in many applications because they exhibit higher yield strengths and can create greater damping forces.

The particle component compositions typically include micron-sized magnetic-responsive particles. In the presence of a magnetic field, the magnetic-responsive particles become polarized and are thereby organized into chains of particles or particle fibrils. The particle chains increase the apparent viscosity (flow resistance) of the fluid, resulting in the development of a solid mass having a yield stress that must be exceeded to induce onset of flow of the magnetorheological fluid. The particles return to an unorganized state when the magnetic field is removed, which lowers the viscosity of the fluid.

Magnetorheological fluids generally contain a carrier fluid that is an organic fluid, or an oil-based, i.e. hydrophobic fluid. Suitable carrier fluids that can be used include natural fatty oils, mineral oils, polyphenylethers, dibasic acid esters, neopentylpolyol esters, phosphate esters, synthetic cycloparaffins and synthetic paraffins, synthetic unsaturated hydrocarbon oils, monobasic acid esters, glycol esters and ethers, silicate esters, silicone oils, silicone copolymers, synthetic hydrocarbons, and mixtures or blends thereof. Examples of other suitable fluids include silicone oils, silicone copolymers, white oils, hydraulic oils, and transformer oils. Hydrocarbons, such as mineral oils, paraffins, cycloparaffins (also known as naphthenic oils) and synthetic hydrocarbons are the preferred classes of carrier fluids. The synthetic hydrocarbon oils include those oils derived from oligomerization of olefins such as polybutenes and oils derived from high alpha olefins of from 8 to 20 carbon atoms by acid catalyzed dimerization and by oligomerization using trialkyl aluminum compounds as catalysts. The carrier fluids utilized in the present invention can be prepared by methods well known in the art and many are commercially available, such as Durasyn® PAO and Chevron Synfluid PAO.

The MR fluids of the present invention can contain various additives known to the art and to the literature such as one or more of an anti-friction agents, anti-wear agents, extreme pressure agents, anti-oxidant agents, various surfactants, thixotropes, or viscosity modifiers, and the like. Depending upon desired end uses, the amount of each type of agent can vary such as from about 0.1 to about 3 parts by weight based upon 100 total parts by weight of the MR fluid. The total amount of all such additives is desirably from about 1 to about 5 parts by weight and preferably from about 2 to about 4 parts by weight per 100 total parts by weight of the MR fluid.

However, it is not an aspect of the present invention to use as an additive a fluorocarbon grease to provide anti-settling characteristics to the MR fluid since the above described invention does not have anti-settling problems. Thus, the present invention is free from any fluorocarbon greases, that is, contains less than about 0.01 parts by weight of desirably less 0.005 parts by weight and preferably no parts by weight of any fluorocarbon grease per 100 parts by weight of MR fluid.

Of the various additive compounds, particularly suitable compounds are an organomolybdenum, an organothiophosphorus, or a combination of the two compounds. Suitable organomolybdenum compounds can be a complex whose structure includes at least one molybdenum atom bonded to or coordinated with at least one organic moiety. The organic moiety can be, for example, derived from a saturated or unsaturated hydrocarbon such as alkane, or cycloalkane; an aromatic hydrocarbon such as phenol or thiophenol; an oxygen-containing compound such as carboxylic acid or anhydride, ester, ether, ketone or alcohol; a nitrogen-containing compound such as amidine, amine or imine; or a compound containing more than one functional group such as thiocarboxylic acid, imidic acid, thiol, amide, imide, alkoxy or hydroxy amine, and amino-thiol-alcohol. The precursor for the organic moiety can be a monomeric compound, an oligomer or polymer. A heteroatom such as ═O, —S, ≡N also can be bonded to or coordinated with the molybdenum atom in addition to the organic moiety.

A particularly preferred group of organomolybdenums is described in U.S. Pat. No. 4,889,647 and U.S. Pat. No. 5,412,130, with the latter describing heterocyclic organomolybdates that are prepared by reacting diol, diamino-thiol-alcohol and amino-alcohol compounds with a molybdenum source in the presence of a phase transfer agent. U.S. Pat. No. 4,889,647 describes an organomolybdenum complex that is prepared by reacting a fatty oil, diethanolamine and a molybdenum source. An organomolybdenum that is prepared according to U.S. Pat. No. 4,889,647 and U.S. Pat. No. 5,412,130 is available from R. T. Vanderbilt Co. under the tradename Molyvan® 855.

Organomolybdenums that can be useful are described in U.S. Pat. No. 5,137,647 that describes an organomolybdenum that is prepared by reacting an amine-amide with a molybdenum source, U.S. Pat. No. 4,990,271 that describes a molybdenum hexacarbonyl dixanthogen, U.S. Pat. No. 4,164,473 that describes an organomolybdenum that is prepared by reacting a hydrocarbyl substituted hydroxy alkylated amine with a molybdenum source, and U.S. Pat. No. 2,805,997 that describes alkyl esters of molybdic acid. All of the above patents relating to organomolybdenum compounds are hereby fully incorporated by reference.

The organomolybdenum compound that is added to the magnetorheological fluid preferably is in a liquid state at ambient room temperature and does not contain any particles above molecular size.

Various organothiophosphorus compounds that can be utilized can have the formula

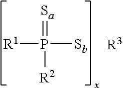

wherein R¹ and R² each individually have a structure represented by:

Y—(C)(R⁴)(R⁵))ₙ—O_{w}—

wherein Y is hydrogen or a functional group—containing moiety such as an amino, amido, imido, carboxyl, hydroxyl, carbonyl, oxo or aryl;

n is an integer from 2 to 17 such that C(R⁴)(R⁵) is a divalent group having a structure such as a straight-chained aliphatic, branched aliphatic, heterocyclic, or aromatic ring;

R⁴ and R⁵ can each individually be hydrogen, alkyl or alkoxy; and

w is 0 or 1.

R³ can be a metal ion such as molybdenum, tin, antimony, lead, bismuth, nickel, iron, zinc, silver, cadmium or lead or a nonmetallic moiety such as hydrogen, a sulfur-containing group, alkyl, alkylaryl, arylalkyl, hydroxyalkyl, an oxy-containing group, amido or an amine. Subscripts a and b are each individually 0 or 1, provided a+b is at least equal to 1 and x is an integer from 1 to 5 depending upon the valence number of R³.

A detailed description of such organothiophosphorus compounds are set forth in U.S. Pat. No. 5,683,615, and is hereby fully incorporated by reference.

Other suitable compounds include those discussed in U.S. Pat. Nos. 7,217,372; 6,203,717; 5,906,676; 5,705,085; and 5,683,615, all hereby fully incorporated by reference.

The total amount of the one or more organomolybdenum compounds and the one or more organothiophosphorus compounds is generally from about 0.1 to about 3.0 and preferably from about 0.2 to about 2.0 parts by weight per every 100 total parts by weight of the MR fluid.

Preparation of MR Fluids.

MR fluids with various formulations were made using the following general process. Carrier fluids (a mixture of synthetic hydrocarbon and fatty ester oils) were measured into a stainless-steel beaker and mixed. An organoclay was added to oils while dispersing with a rotor-stator at 2500 revolutions per minute (RPM) (standard clay level) or 3600 RPM (low clay), and clay activator was added immediately after clay addition was complete. The resulting mixture was dispersed for 10 minutes at 2500 RPM (standard clay level) or for 20 minutes at 3600 RPM (low clay level). Iron powder was added and dispersed at 4100 RPM (low clay level) or 3600 RPM (standard clay level) for 10 minutes. The suspension thickened substantially and warmed to approximately 50° C. during this dispersion step. The rotor-stator speed was reduced to 3600 RPM (low clay level) or to 2500 RPM (standard clay level) and the additives (friction modifier (FM), anti-wear additive (AW), and/or polyol) were mixed in for about 5 minutes.

Bomb Tests.

Bomb tests were conducted by placing 100 mL of MR fluid into a 1 liter stainless steel bomb, sealing the bomb under ambient air pressure, and placing the bomb in an oven at 200° C. for 72 hours.

Measurement of Settling.

Short-term settling was determined by measuring the percentage of clear liquid layer that formed upon standing for 24 hours at room temperature in a clear plastic vial. Long term percent clear layer and sediment hardness were determined after thermal cycling of a 400-mL sample from −20° C. to 125° C. for seven days in a sealed paint can.

Viscosity Measurement.

Viscosity was measured at 40° C. using a TA Instruments AR-2000 rheometer with a Couette geometry. The sample was equilibrated at 40° C., pre-sheared at 100 s⁻¹ for 5 minutes, then the shear stress was measured as the shear rate was ramped up from 0 to 1200 s⁻¹ and back down to 0 s⁻¹ over 20 minutes. Viscosity and yield stress were determined as the slope and y-intercept, respectively, of the down curve from 800 to 1200 s⁻¹.

An initial experiment to test the dispersion ability of the polyols of the present invention in MR fluids was conducted using the polyol of Trial 3 in Table 1. An MR fluid was prepared as a control sample, and a test sample was prepared using the same formulation but replacing the friction modifier and anti-wear agents with the polyol (entries 1 and 2 in Table 4). During preparation of the polyol-containing MR fluid, it was observed that the thick slurry of iron and clay became substantially thinner upon addition of the polyol. A similar thinning of MR fluids is observed generally upon addition of common friction modifiers to iron suspensions. Overnight and long-term settling of these fluids were comparable, indicating that the polyol does not significantly interfere with the action of the organoclay suspension aid.

Several MR fluid formulations were prepared in which the usual friction modifier was replaced with one of five different polyols at the same weight percentage. The general formulation is shown in Table 4. Fluids were tested for short- and long-term settling, and their 40° C. viscosities were measured before and after the bomb test. The polyols used and the test results are summarized in Table 5.

TABLE 4
General MR Fluid Formulation

| Component | Wt % |
|---|---|
| Iron Powder | 76.40 |
| Ester Oil | 3.44 |
| Organoclay | 0.99 |
| Clay activator | 0.21 |
| AW additive (Anti wear) | 0.48 |
| Polyol of the invention | 0.57 |
| Synthetic hydrocarbon oil | 17.91 |
| Total | 100.00 |

TABLE 5
Settling and viscosity data summary for MR fluids with Guerbet polyols

| | | | | | Long- | Long- | | Fresh | | Post-test | |
|---|---|---|---|---|---|---|---|---|---|---|---|
| | | | | | term | term | Fresh | 40 C | | 40 C | |
| | | | Vol % | | Clear | sediment | 40 C | Yield | Post-test | Yield | |
| | | Polyol | Polyol | 24-hr | layer | Hardness | Visc | Stress | 40 C Visc | Stress | % Visc |
| | Description | ID | (d = 1) | settling | (%) | (N) | (Pa-s) | (Pa) | (Pa-s) | (Pa) | Increase |
| 1 | ½ clay and ½ adds | None | 0 | 2.1 | 43 | 2.3 | 0.045 | 3 | 0.100 | 5 | 122 |
| 2 | ½ clay polyol replacing | Trial 3 | 1.0 | 2.4 | 36 | 4.1 | 0.053 | 3 | 0.060 | 2 | 14 |
| | additives | | | | | | | | | | |
| 3 | Control | None | 0 | 1.9 | 18 | 1.9 | 0.063 | 3 | 0.194 | 3 | 208 |
| 4 | Control without FM | None | 0 | n/a | n/a | n/a | 0.055 | 6 | 0.158 | 7 | 187 |
| 5 | Full clay, polyol replacing FM | Trial 10 | 1.51 | 2.1 | 22 | 3.9 | 0.079 | 4 | 0.141 | 1 | 78 |
| 6 | Full clay, polyol replacing FM | Trial 11 | 1.51 | 1.8 | 19 | 4.9 | 0.076 | 8 | 0.139 | 2 | 82 |
| 7 | Full clay, polyol replacing FM | Trial 12 | 1.51 | 2.4 | 17 | 5.6 | 0.076 | 8 | 0.173 | 5 | 129 |
| 8 | Full clay, polyol replacing FM | Trial 13 | 1.51 | 2.1 | 18 | 6.3 | 0.073 | 8 | 0.252 | 25 | 247 |
| 9 | Full clay, polyol replacing FM | Trial 13 | 1.51 | 1.6 | 16 | 6.2 | 0.074 | 7 | 0.188 | 10 | 153 |
| 10 | Full clay, 10% polyol w/AW | Trial 11 | 0.151 | | 20 | 4.4 | 0.063 | 3 | 0.136 | 5 | 115 |
| 11 | Full clay, 10% polyol w/AW | Trial 13 | 0.151 | | 18 | 4.9 | 0.062 | 3 | 0.146 | 6 | 135 |
| 12 | Full clay, 10% polyol w/AW | Trial 13 | 0.151 | | 17 | 4.4 | 0.062 | 4 | 0.147 | 8 | 138 |
| 13 | Full clay, 10% polyol w/AW | Control | 1.51 | 1.8 | 15 | 2.9 | 0.076 | 7 | 0.126 | 1 | 66 |
| 14 | Full clay, mono-ol w/AW | Control | 1.51 | 1.6 | 13 | 2.3 | 0.061 | 4 | 0.118 | 2.5 | 92 |

FM = Friction modifier
AW = anti-wear additive

Various MR fluids were subjected to a bomb test to assess their thermal stability. Commercial LORD MR fluids, as well as formulations prepared with a variety of commercial anti-oxidant additives, were observed to have a viscosity increases of about 200-300% after the bomb test. The control fluid viscosity more than doubled (122% increase), but the viscosity of the MR fluid with polyols of the present invention increased by only 14% (see data summary in Table 5).

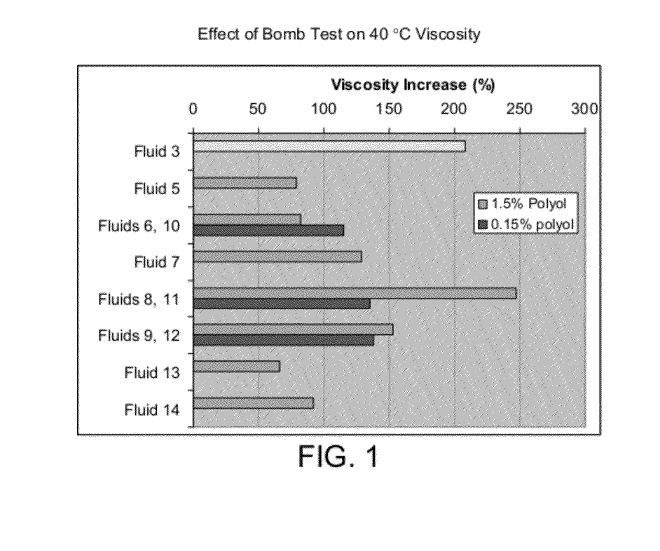

The viscosity increase after bomb test was also substantially higher than in the first experiment, with all polyols except Trial 13 giving lower increases than the control fluid (entry 4 in Table 5). Also see FIG. 1. Observing the trends in fluids with polyols from the half-factorial DOE with Pripol, diol:mono-ol ratio, and catalyst type as factors (entries 6-9), fluids with the lower mono-ol:diol ratio (1.5 mole:mole, entries 6 and 7) had the lowest viscosity increase and the lowest sediment hardness values. A secondary effect was that polyols with the higher Pripol level (0.45 weight fraction) with the same diol:mono-ol ratio (entries 6 and 9) gave lower viscosity increase and lower sediment hardness in thermally aged MR fluids. Overall, most polyols gave an advantage over the usual additives in terms of MR fluid thermal stability.

All polyols were observed to have a thinning effect on the iron suspension at low levels, even before all polyol was added. Addition of the full amount of polyol seemed to thicken the fluid slightly, as shown in the somewhat higher 40° C. viscosity as compared to the control fluid. Short- and long-term clear layer after settling were comparable to the control, but the sediment hardness after long-term testing was significantly higher than control.

Without being bound by theory, it is believed that the polyol interacts with the organoclay and/or iron particle surfaces in a similar way that the friction modifier does and thus modifies the interparticle forces. The result of this modification is to lower the interparticle forces sufficiently to provide low dynamic viscosity but still maintain sufficient force under static conditions to maintain good settling properties. Under conditions of the bomb test, the normal friction modifier degrades and its effect on the interparticle forces is decreased so that the fluid viscosity increases. The polyols have higher thermal stability and are not as fully degraded, therefore maintaining their effect on the interparticle forces. Variations in performance between the different polyols are likely related to solubility differences and/or differences in the strength of interaction with the particle surfaces.

Drilling Fluids

A characteristic drilling fluid comprises those enumerated in U.S. Pat. No. 6,806,235, U.S. Pat. No. 6,716,799, and U.S. Pat. No. 5,869,434, hereby fully incorporated by reference. With respect to drilling fluids, the polyols or polyol compositions of the present invention have shown improvement with respect to one or more of the following properties; lubricity, reduced fluid loss, and favorably impact on rheology in both water and hydrocarbon based fluids. An analysis of the above and other patents was undertaken to better understand the current problems that are being addressed by additives to oil-based and synthetic drilling fluids. The three main problem areas being addressed by inventions in this field are “rheology”—the non-newtonian flow characteristics at various shear rates and temperatures, “fluid loss”—loss of liquid portions of the drilling fluid to porous or fractured geological formations, and “emulsification”—as most oil-based fluids contain an aqueous portion dispersed in the oil and all systems will become contaminated by ground water. Further, deeper and laterally drilled wells result in greater friction to the drilling string promoting a migration to more lubricious drilling fluids such as provided by oil based muds and synthetic muds. Often, these extreme wells also encounter higher temperatures severely stressing the chemical stability of standard additives used in drilling fluids.

The 200° C. bomb test results illustrate that stability in hot wells is likely to be realized. The polyols of the present invention represent a significant improvement over the mono alcohols currently employed in drilling fluids due to the polyols' lower vapor pressure at elevated temperatures and their multiple attachment points. The latter characteristic further imparts wear protection if adsorbed as a boundary layer on a metal surface.

Industrial/Automotive Lubricating Fluids

Industrial and/or automotive lubricating fluids generally contain a lubricating oil, various antioxidants, various antiwear and extreme-pressure additives, friction modifiers, surfactants, dispersants and other additives as necessary.

Improvements in lubricant additives for internal combustion engines are driven by 1) improved fuel efficiency (government mandated), 2) emissions (harm to catalytic converters), and a more distant 3) oil life span.

Fuel Efficiency

The largest contribution of engine oils to fuel efficiency is by reducing friction, a property addressed by several patents in the early 1980's. Proposed solutions heretofore having included U.S. Pat. No. 4,228,020, for example, a combination of graphite and a di-lower alkyl hydrocarby phosphonate. However, graphite imparts an opaque, black color to the oil generally an attribute of dirty, used oil. U.S. Pat. No. 4,243,539 alleges that N-hydroxymethyl aliphatic hydrocarbylamides reduces friction in internal combustion engines. U.S. Pat. No. 4,293,432 relates to a reaction product of a fatty acid and a monoethanolamine optionally combined with a di-lower alkyl hydrocarbylphosphonate. More recently, U.S. Pat. No. 7,989,408 describes a new base oil mixture combined with a well studied class of friction additives, mono-esters of fatty acids.

Emissions

Recent standards set for gasoline engines relate to reducing the level of phosphorous as this element has been identified as a catalyst poison in emission control systems. Future standards are expected to further reduce sulfur emissions that poison catalysts for removing nitrogen oxides from exhaust fumes. The principal source of these two elements is the most widely used anti-wear agent zinc dialkyl dithiophosphate (ZDDP). Any additive that can allow removal or lowering the concentration of ZDDP used would contribute significantly to this goal. U.S. Pat. No. 7,875,580 describes one such approach.

Oil Life Span

Industry movement to less frequent oil changes are generally limited by oxidative degradation of either the base oil or essential additives. Additives with greater resistance to oxidative and hydrolysis processes in combination with known free radical inhibitors are attractive. Chemical structures known to be susceptible to these processes include ethers, esters, olefins, ketones, among others. Polyols of the current invention provide an all carbon backbone.

Modifying the additive package by utilizing the polyols or polyol compositions of the present invention is thought to have relatively minor effects on the first, but have substantial impact on the other two.

Adhesives—Coatings—Greases

Polyols of this invention can be used without further derivatization to replace or partially replace other commonly used polyols in adhesives, coatings, and greases such as polyurethane adhesives and coatings, cationically cured epoxy adhesives and coatings, polyol coatings cured by methylol melamine, tetramethylol glycoluril, and other methylolated ureas derivatives, coatings cured by transesterification (see U.S. Pat. No. 4,749,728), as well as long chain hydrocarbons and other greases, known to the art as well as to the literature.

The polyols can be converted into other functionality by commonly known methods. Such derivatives include glycidyl ethers, vinyl ethers, alkyl ethers, propenyl compounds, (meth)acrylic esters, and the like. These derivatized products can be used analogously to similar materials, but have the advantage of possessing no or fewer hydrolyzable linkages.

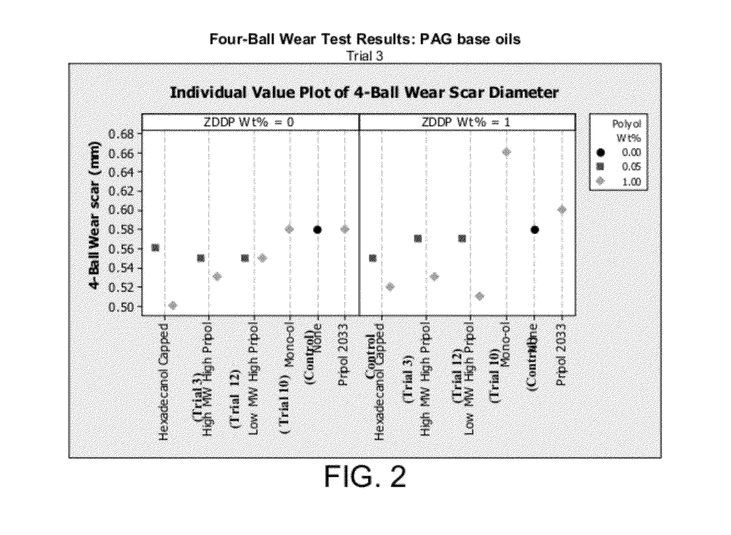

Various polyols of the present invention were utilized as additives in mineral oil as well as in polyalkylene glycols with regard to a four-ball wear tests, ASTM D-2266 and D2596. The results thereof with regard to scar diameter and coefficient of friction are set forth in FIGS. 2-5.

The amount of the polyols of the present invention in various compounds such as base oils, MR fluids, drilling fluids, engine lubricants, adhesives, coatings and greases can vary such as from about 0.1 to about 5, desirably from about 0.1 to about 3, and often from about 0.2 to about 2 parts by weight per 100 parts by weight of the compound.

### EXAMPLES

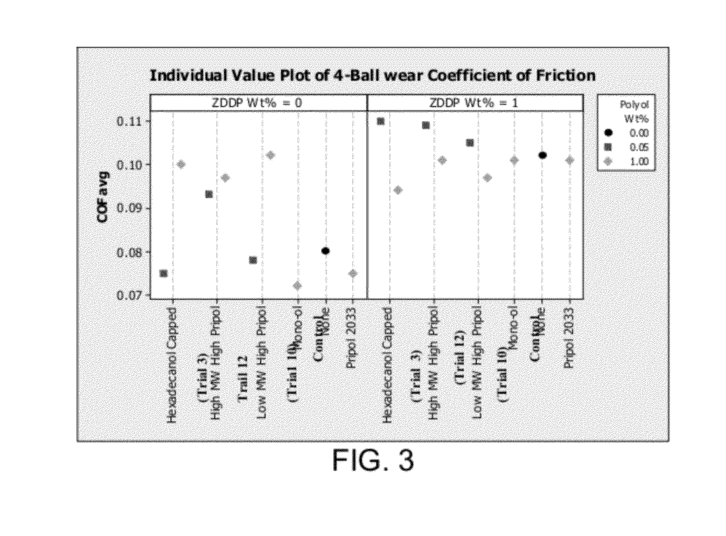

Samples of poly(alkylene glycol) base oil UCON HB-55 oil from Dow) were prepared with either 0.05% or 1% of three polyol additives (Trials 3, 10, and 12), as well as the control mono-ol and Pripol 2033 used in preparing polyols 10 and 12. Additional samples were prepared containing polyol, mono-ol or Pripol 2033 and 1% of the automotive oil additive zinc dialkyl dithiophosphate, ZDDP (e.g. Lubrizol® 1394). The base oil with no additives was also tested as a control. All samples were tested in the four-ball wear test ASTM D-2266, in which the diameter of the wear scar after the test and the average coefficient of friction during the test are measured. A smaller wear scar and/or a lower coefficient of friction are indicators of better lubrication properties. FIG. 2 summarizes the wear scar diameters for the various samples. As shown in FIG. 2, the three polyol additives at 1% have a smaller wear scar than the pure PAG oil or the two control samples. The improvement was even greater over the mono-ol in the presence of ZDDP. No improvements in coefficient of friction were observed. FIG. 3 summarizes the coefficients of friction for the same samples. In the absence of ZDDP, the three polyols actually increase the coefficient of friction over the control samples. However, in the presence of ZDDP, which causes an increase in friction of the base oil and the two controls, the polyols at 1% give a slight improvement. Taken together, the data show that improve the lubricity of the PAG, especially in the presence of ZDDP.

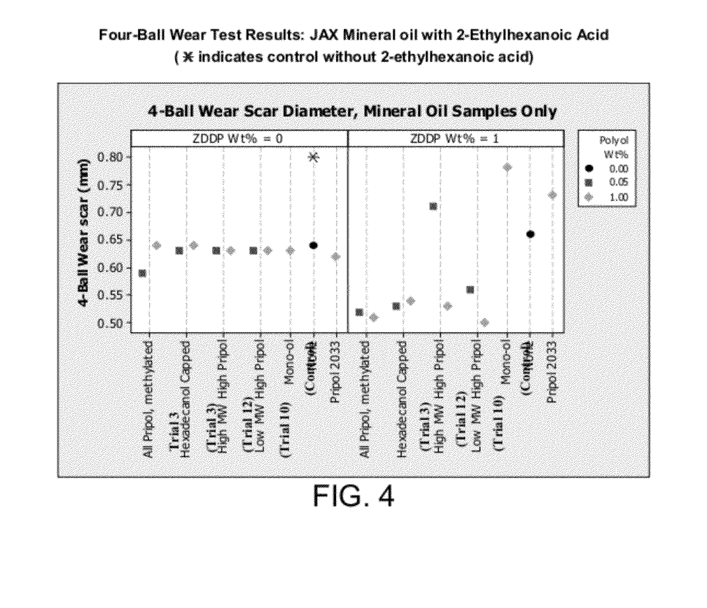

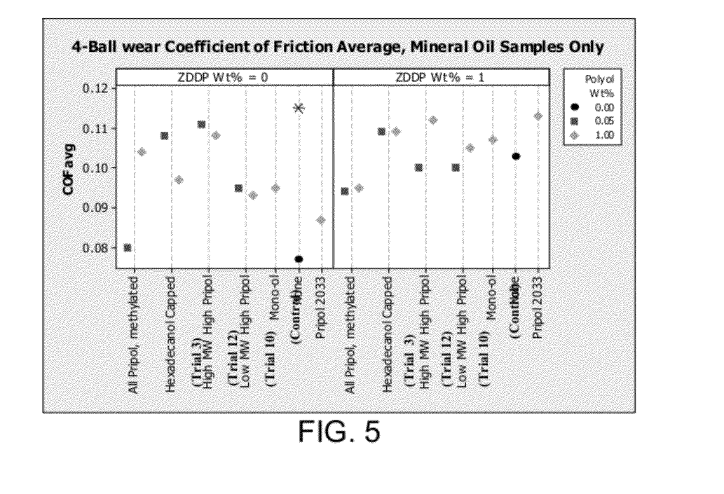

Similarly to the above, samples were also prepared in mineral oil (JAX white mineral oil obtained from Ethyl Corporation) with and without additional ZDDP, and lubricity properties were tested in the four-ball wear test ASTM D-2266. To promote solubility of the polyols in mineral oil, it was necessary to add about 5% 2-ethylhexanoic acid. The wear scar diameters, shown in FIG. 4, show that the polyols had no effect in the absence of ZDDP but improved the wear scar in the presence of ZDDP. Data for the coefficients of friction are shown in FIG. 5. In the absence of ZDDP the coefficients of friction are higher with the polyols, but in the presence of ZDDP the friction is either unchanged or slightly improved, as in the case of Trial 17. Thus, as observed in the PAG oils, the polyols show the ability to improve the wear properties of lubricants containing ZDDP, an important property given that ZDDP is a ubiquitous additive in automotive lubricants.

### Evaluation of Undiluted Polyols by the ASTM D2596 Extreme Pressure (4-Ball Wear Test)

Five polyols were evaluated without dilution or modification from their initial synthesis. Two fully formulated commercial grease samples were also tested as controls. The results are tabulated below.

TABLE 6

| | | Load Wear | Weld |
|---|---|---|---|
| | | Index | Point |
| Trial | Description | (Kgf) | (kg) |
| 6 | 1-Hexadecanol capped 1,6-hexanediol | 34.61 | 160 |
| 14 | ½ 3-pyridinemethanol, | 63.57 | 250 |
| | ½ 1-hexadecanol terminated | | |
| 12 | Design: high Pripol; high | 50.67 | 200 |
| | diol/monol; ZnMo | | |
| 9 | n-Butanol capped 1,10-decanediol | 64.07 | 250 |
| 15 | “oxo” 9793-39B, methylated | 35.64 | 250 |
| Comp. | Walmart “SuperTech Grease” General | 22.45 | 160 |
| Ex. 1 | purpose | | |
| Comp. | Walmart “Multi-duty Grease” | 49.81 | 315 |
| Ex. 2 | | | |

Three of the five polyols tested exceeded the load wear index of the high performance control grease. Two of these also had weld points that are moderately high at 250 kg.

Evaluation of Drilling Fluids at an Independent Test Facility

Series 1—1 wt % Polyol Added to a (a Polyalphaolefin Fluid)

Four polyols and a control mono-ol, 2-tetradecyloctadecan-1-ol, were added to the base fluid as a post-addition using a using a Silverson mixer. Initial tests were performed on base fluid and each of the five blended samples. Rheological parameters were measured using a Fann 35 viscometer at 65.6° C., and HTHP filtration was performed at 148.9° C. The filtration tests were reported as milliliters collected after 30 minutes. Lubricity tests were conducted on each of the six fluids. Separate samples of the same material were rolled at 200° C. for 24 hours, cooled to room temperature, and finally mixed on a lab dispersator for ten minutes. The preceding tests were repeated on the heat aged samples.

Lubricity Test Procedure

1. The test ring and metal block of the Lubricity Meter was carefully washed with a mild detergent, than cleaned with isopropyl alcohol before each test.
2. The meter was calibrated with distilled water over a 70 minute period for standardization of the block and ring apparatus.
3. The Lubricity Meter is a device that uses a rotating metal ring against a mated, metal block as contact surfaces. The test fluid is placed in a metal bowl, then used to immerse the metal ring and block. After turning on the machine motor and adjusting the rotation to 60 rpm, a torque meter is used to apply a load of 150 inch-lbs to the rotating metal ring. A reading from the gauge indicates the coefficient of friction of the sample. This reading is taken after one minute, three minutes, and five minutes. The average coefficient of friction from these readings is calculated.

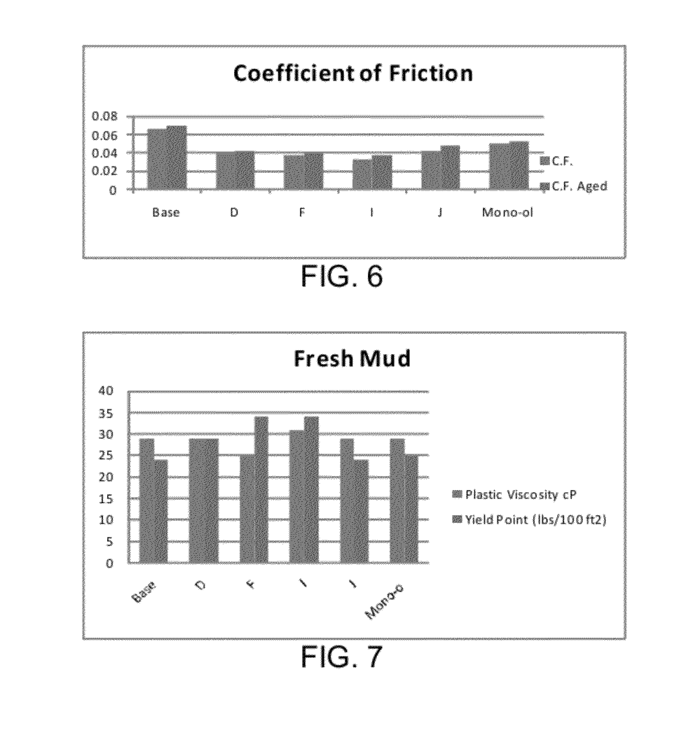

FIGS. 6, 7, and 8 summarize the results obtained. The polyols designated D, F, I, and J were polyols from Trials 10 replicate, 11 replicate, 16, and 17, respectively. See Table 7.

TABLE 7

| Label | Trial No., Table 1 |
|---|---|
| A | 6 |
| B | 12 |
| C | 13 |
| D | 1st rep. 10 |
| E | 15 |
| F | 1st rep. 11 |
| G | 19 |
| I | 16 |
| J | 17 |
| K | 8 |
| L | Mono-ol |
| M | 11 |
| N | 2nd rep. 11 |
| 38 | 20 |
| 13A | 14 |

With respect to FIG. 6, all additives caused a significant decrease in the coefficient of friction with the polyols yet being better than the mono-ol control. The base fluid and all the additives were stable to 24 hour aging at 200° C. A lower coefficient of friction when extended to an extended reach well would result in greater drilling distance or reduced power requirements of the drilling motor.

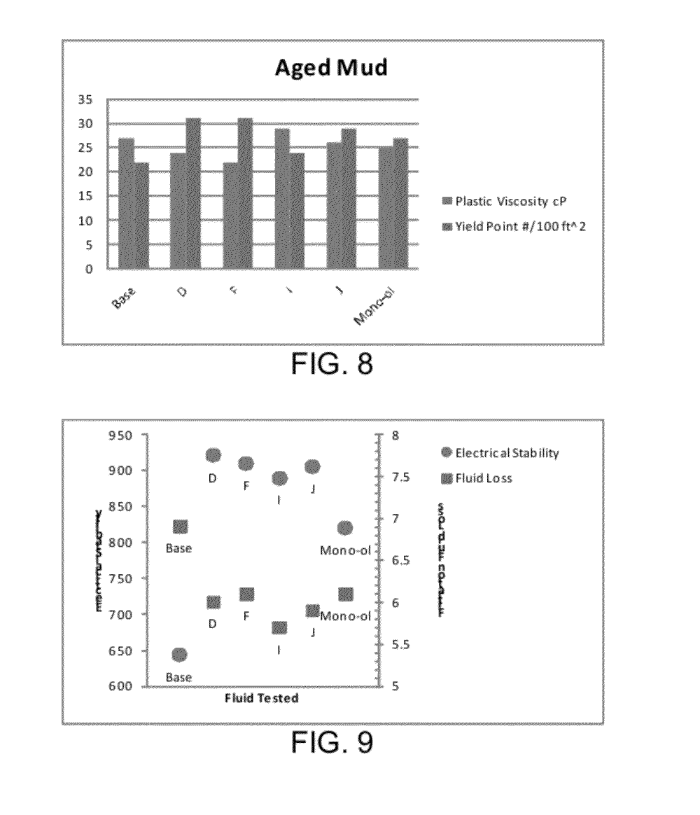

FIGS. 7 and 8 show drilling fluids are preferred that have as low as possible plastic viscosity and a targeted yield point. Usually these properties move together. Some of the polyols give improved yield point and yield point retention on aging while also showing equal or reduced plastic viscosity. Polyol formulations D and F appear particularly attractive.

Fluid loss is the tendency for a drilling fluid to penetrate into porous rock formations. This is desired to be as low as possible. As shown in FIG. 9, all additives reduced the loss, with most being better than the mono-ol control. Electrical stability is a measure of the inverse emulsion fluids to breakdown in the presence of an electric field. A higher number is better. While all additives made a large improvement relative to the base fluid, the polyols were all much better than the mono-ol control.

The polyols were designated D, F, I, and J. (See Table 7) These designations corresponded to a remake composition that was compositionally the same as Trial 10, Table 2, a remake composition that was compositionally the same as Trial 11, Table 2, a polyol made with Pripol 2033 as the only diol and terminated with 1-octadecanol, Trial 16, Table 2, and a replicate of Trial 16 that had been post-treated with dimethyl sulfate (converts the carboxylate groups to methyl esters), Trial 17, Table 2.

Series 2—1 wt. % Polyol added to an Escaid 110 fluid (a mineral oil base)

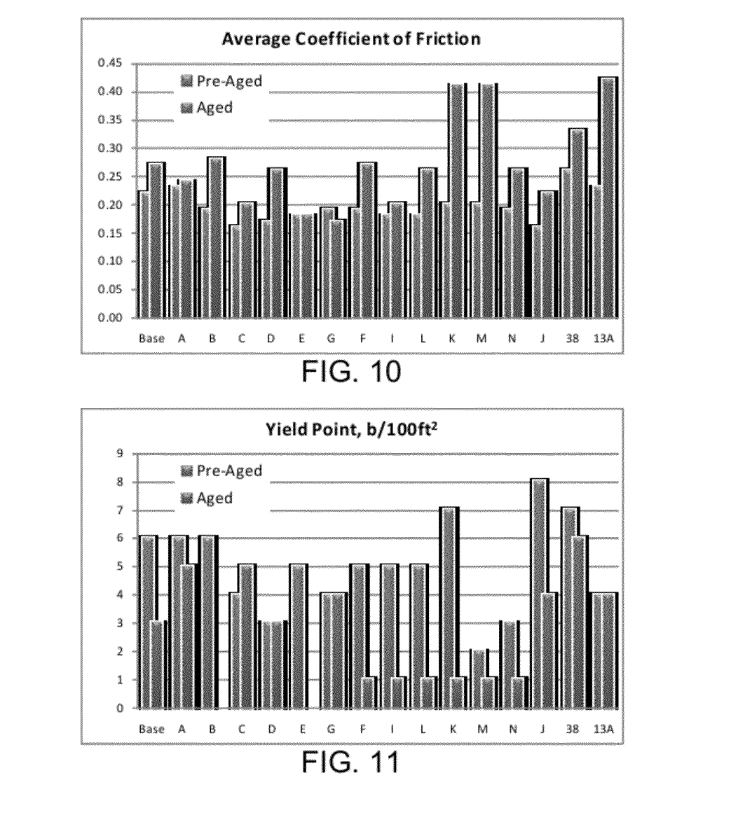

Fourteen polyols and the same mono-ol used in the first series were added to the base fluid as a post-addition using a Silverson mixer. Initial tests were performed on base fluid and each of the fifteen blended samples. Rheological parameters were measured using a Fann 35 viscometer at 65.6° C., and HTHP filtration was performed at 148.9° C. The filtration tests were reported as milliliters collected after 30 minutes. Lubricity tests were conducted on each of the six fluids. Separate samples of the same material were rolled at 200° C. for 24 hours, cooled to room temperature, and finally mixed on a lab dispersator for ten minutes. The preceding tests were repeated on the heat aged samples. FIGS. 10 through 14 summarize the results obtained. The polyols were as designated as shown in Table 7. NOTE: The base fluid was not properly formulated as requested to have an electrical stability of greater than 600 V. For most of the fluids, one or more properties were severely compromised by heat aging, possibly as a consequence of this mis-formulation, but possibly because this mineral based fluid is just inherently less robust.

Focusing only on the pre-aged data, FIG. 10 shows that most of the polyols gave an improvement in the coefficient of friction with a few being even better than the mono-ol control additive.

FIG. 11 shows various examples of pre-aged and aged yield points.

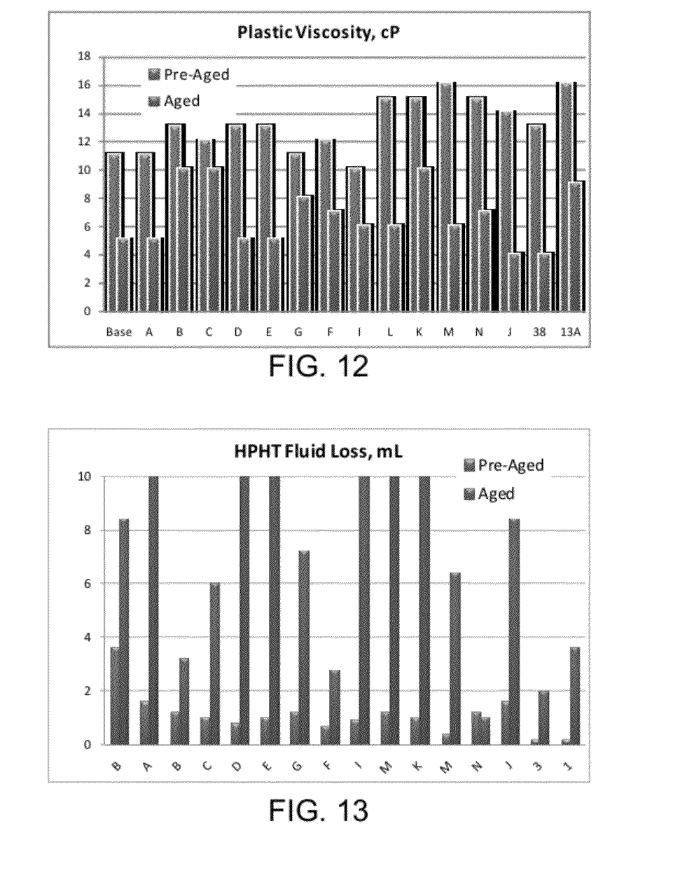

FIG. 12 shows that in this base oil, the attractive balance of high yield point and low plastic viscosity seen in series 1 was not achieved.

FIG. 13 shows that the fluid loss was markedly lower for all of the polyol containing samples before heat aging and five of them showed equal or better fluid retention after heat aging than the base fluid showed before heat aging.

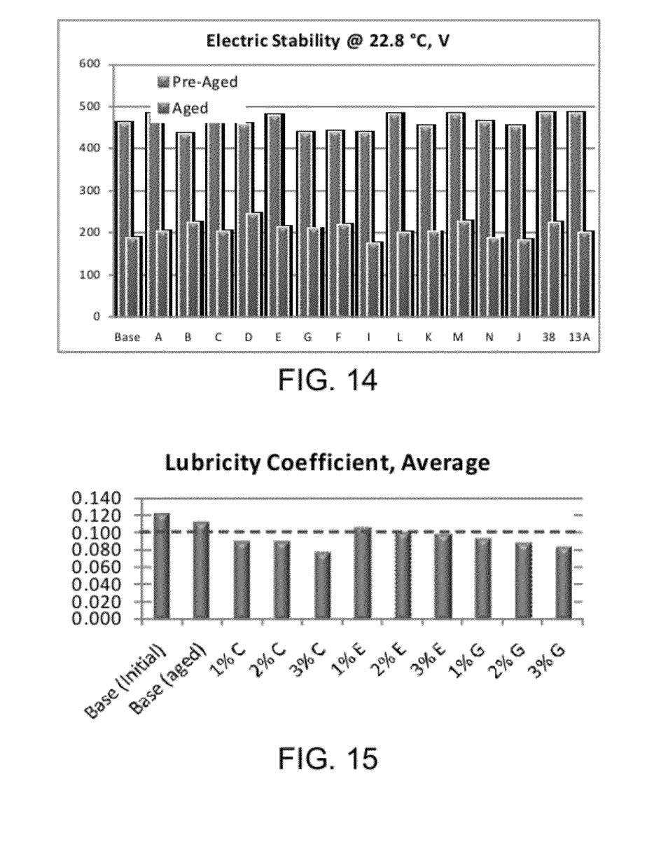

The large drop in electrical stability after heat aging in FIG. 14 shows that none of the polyols could overcome the poor stability of the base fluid at this very high temperature of 200° C.

Series 3—Polyol Added to an Escaid 110 Fluid (a Mineral Oil Base)

The same test procedures were used as in Series 2, but the base formula had been adjusted to give a higher electrical stability initially through standard formulation adjustments, tested at 48.9° C., and the heat aging was done at 121.1° C. for 16 hours. While the base fluid was tested before and after heat aging, the polyol containing samples were only tested after heat aging. Only three polyols, C, E, and G, which had had the lowest coefficient of friction in Series 2, were evaluated, but each was tested at three levels, 1 wt. %, 2 wt. %, and 3 wt. %. FIGS. 15 through 18 summarize the results.

The dashed line in FIG. 15 marks a lubricity value of 0.10, a target, ceiling value. A lower value was desired. All the polyol containing samples except the lowest loading of polyol E were under the target value.

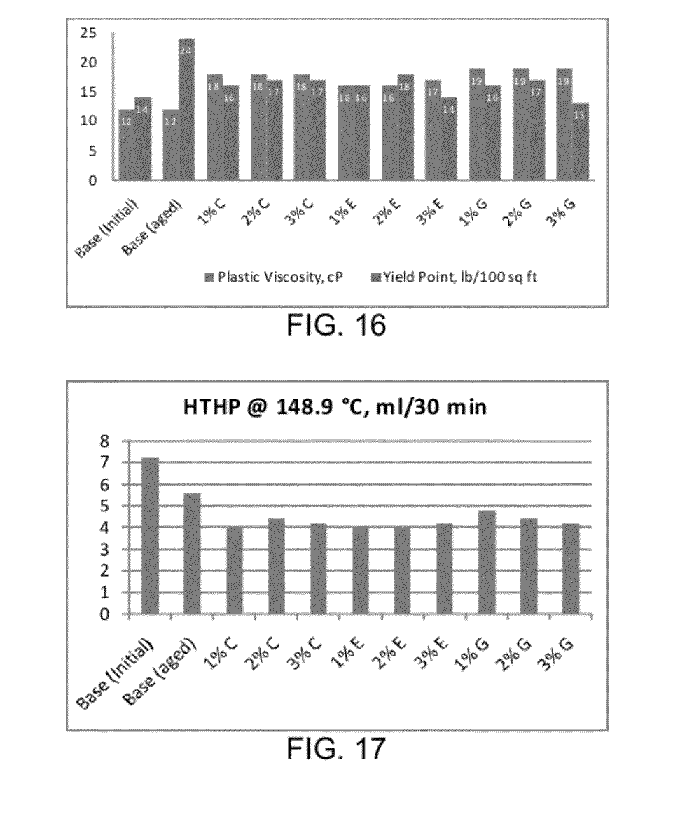

FIG. 16 shows that no particular benefit was seen in the rheological results relative to the control.

FIG. 17 shows that all three polyols gave improved filtration resistance relative to the base fluid, but there was little difference with polyol type or level

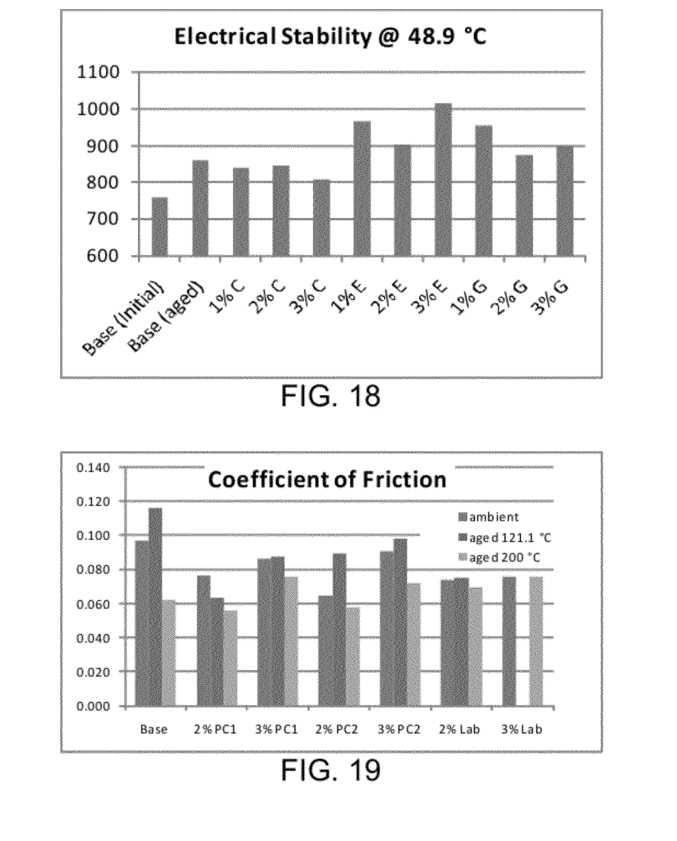

FIG. 18 shows amore stable base fluid, relative to that used in series 2, was achieved with polyols E and G.

Series 4—Polyol Added to an Escaid 110 Fluid (a Mineral Oil Base)

For this round of testing, polyol C was prepared twice at a 50 L pilot scale. These two batches and the original laboratory batch were evaluated at 2 wt. % and 3 wt. % as for Series 2, but with separate samples both aged at 121.1° C. for 16 hours and aged at 200° C. for 24 hours. None of the fluids were stable to the higher aging temperature. The results are summarized in FIGS. 19 through 23.

The coefficient of friction values in FIG. 19 generally shows slightly lower values at 2% than 3%, all being lower than the base fluid and lower than the target ceiling value of 0.1 for both ambient and after the 121° C. aging.

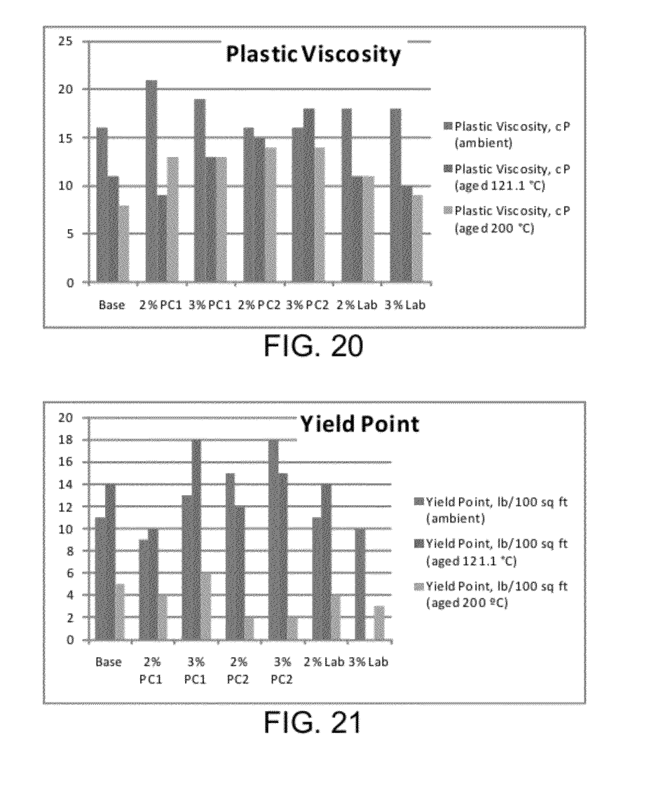

FIGS. 20 and 21 show no particular trend nor advantage was found in the rheological values of yield point and plastic viscosity. The yield point data after 200° C. shows one of the consequences of failure at the high temperature.

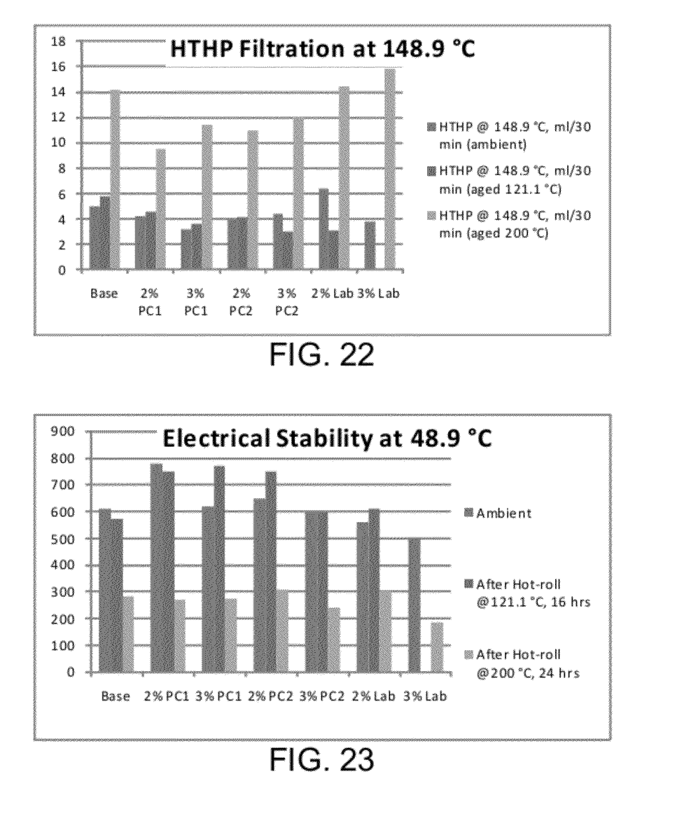

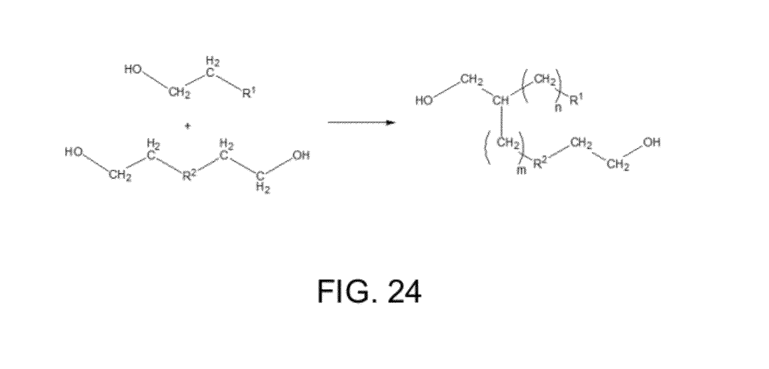

FIG. 22 generally shows the filtration data for the polyol additives were favorably lower than the control and lower at the higher level of additive.

FIG. 23 generally shows the electrical stability was better at the lower level of additive.

The data after heat aging at 200° C. indicates that all samples were severely degraded by this aggressive test.

While in accordance with the patent statutes the best mode and preferred embodiment have been set forth, the scope of the invention is not intended to be limited thereto, but only by the scope of the attached claims.

What is claimed is:

1. A polyol composition comprising:
the reactant of a mono-ol with a diol;
wherein said mono-ol, independently, comprises
a compound having the formula R—CH₂—CH₂—OH wherein R is a linear alkyl, branched alkyl, cyclic alkyl, or heterocyclic alkyl, or any combination thereof; or
a compound having the formula Ar—CH₂—OH wherein Ar is a phenyl, pyridyl, furanyl, m- or p-alkylphenyl, or any other meta or para substituents compatible with the reaction conditions of said mono-ol with said diol; or
a compound having the formula Ar′-Q-CH₂—CH₂—OH where Q is —(CR′₂)—ₙ, wherein n is from 1 to about 10, each R′, independently, is H or R as defined above, and wherein Ar′ is phenyl, pyridyl, furanyl, o-, m-, p-alkylphenyl, o-, m-, or p-phenoxyphenyl, or any other ortho, meta, para substituents compatible with the reaction conditions of the mono-ol with said diol;
wherein said diol, independently, comprises
a compound having the formula R—(CH₂—CH₂—OH)₂ wherein R is a linear alkyl, branched alkyl, cyclic alkyl, or heterocyclic alkyl, or any combination thereof; or
a compound having the formula Ar(CH₂—OH)₂ wherein Ar is m-phenyl, m- or m′- or p- or p′-diphenyl ether, or m- or m′ or p- or p′-diphenylmethane, or any other ortho, meta, or para substituents compatible with the reaction conditions of said mono-ol with said diol; or
a compound having the formula Ar′(-Q-(CH₂)ₖ—CH₂—OH)₂ where each Q, independently, is —(CR″₂)—ₙ, where each n, independently, is 0 or 1 to about 10 and each R″, independently, can be H or R as defined hereinabove, where each k, independently, is 0 or 1, and wherein Ar′ can be a, o-, m-, or p-alkylphenyl, o-, m-, or p-phenoxyphenyl, or any other ortho, meta, or para substituents compatible with the reaction conditions of said mono-ol with said diol; and
wherein said R′, Ar, and Ar′ of said different diol formulas can, independently, be the same or different, than said R, Ar, and Ar′ of said mono-ol.

2. A process for forming a polyol composition, comprising the steps of:
reacting a mono-ol with a diol in the presence of a basic catalyst;
wherein said mono-ol, independently, comprises
a compound having the formula R—CH₂—CH₂—OH wherein R is a linear alkyl, branched alkyl, cyclic alkyl, or heterocyclic alkyl, or any combination thereof; or
a compound having the formula Ar—CH₂—OH wherein Ar is a phenyl, pyridyl, furanyl, m- or p-alkylphenyl, or any other meta or para substituents compatible with the reaction conditions of said mono-ol with said diol; and
a compound having the formula Ar′-Q-CH₂—CH₂—OH where Q is —(CR′₂)—ₙ, wherein n is from 1 to about 10, each R′, independently, is H or each R as defined above, and wherein Ar′ is phenyl, pyridyl, furanyl, o-, m-, p-alkylphenyl, o-, m-, or p-phenoxyphenyl, or any other ortho, meta, para substituents compatible with the reaction conditions of the mono-ol with said diol;
wherein said diol, independently, comprises:
a compound having the formula R—(CH₂—CH₂—OH)₂ wherein R is a linear alkyl, branched alkyl, cyclic alkyl, or heterocyclic alkyl, or any combination thereof; or
a compound having the formula Ar(CH₂—OH)₂ wherein Ar is m-phenyl, m- or m′- or p- or p′-diphenyl ether, or m- or m′ or p- or p′-diphenylmethane, or any other ortho, meta, or para substituents compatible with the reaction conditions; or
a compound having the formula Ar′(-Q-(CH₂)ₖ—CH₂—OH)₂ where each Q, independently, is —(CR′₂)ₙ—, where each n, independently, is 0 or 1 to about 10 and each R″, independently, can be H or R′ as defined hereinabove, where each k, independently, is 0 or 1, and wherein Ar′ can be a, o-, m-, or p-alkylphenyl, o-, m-, or p-phenoxyphenyl, or any other ortho, meta, or para substituents compatible with the reaction conditions, or
wherein R, Ar, and Ar′ of said diol can, independently, be the same or different than said R, Ar, and Ar′ of said mono-ol.

3. The polyol composition of claim 1, containing beta branching and wherein said mono-ol comprises said compound having the formula R—CH₂—CH₂—OH wherein R is a linear alkyl, and
wherein said diol comprises a compound having the formula R—(CH₂—CH₂—OH)₂ wherein R is a branched alkyl having from about 2 to about 38 carbon atoms, or
wherein said diol comprises a mixture of R being branched alkyl and linear alkyl and the linear alkyl has from about 2 to about 10 carbon atoms.

4. The polyol composition of claim 3, wherein said branched alkyl diol has from about 28 to about 36 carbon atoms and the linear alkyl, if used, has 6 to about 8 carbon atoms.

5. The polyol composition of claim 1, comprising a compound having the formula: 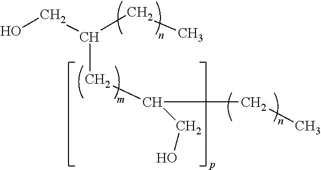
wherein each n, independently, is derived from a linear, aliphatic alcohol having the formula HO—(CH₂)ₐ—CH₃ where a is from 3 to about 41 carbon atoms;
wherein m is derived from a linear aliphatic diol having the formula HO—(CH₂)_{b}—OH where b is from 6 to about 42 carbon atoms; and
wherein n independently is a or a-2 and m=b, b-2, or b-4, and p=1 to about 20.

6. The polyol composition of claim 5, wherein a is from 4 to about 21 carbon atoms, and wherein b is 10 to about 36 carbon atoms.

7. The polyol composition of claim 1, comprising a polyol compound having the formula 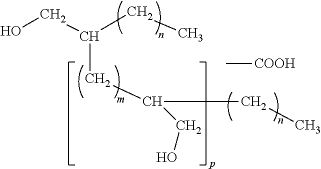
wherein each n, independently, is derived from a linear, aliphatic alcohol having the formula HO—(CH₂)ₐ—CH₃ where a is from 3 to about 41 carbon atoms;
wherein m is derived from a linear, aliphatic diol having the formula HO—(CH₂)_{b}—OH where b is from about 6 to about 42 carbon atoms;
wherein n independently is a or a-2 and m=b, b-2, or b-4, and p is generally 1 to about 20; and
wherein said —COOH replaces a small amount of said —CH₂—OH groups and is bonded to one of said carbon atoms.

8. A magnetorheological fluid comprising the polyol composition of claim 1.

9. A magnetorheological fluid comprising the polyol composition of claim 3, wherein the magnetorheological fluid further comprises at least one or more of the following: a carrier fluid, a antifriction agent, an antiwear agent, an extreme pressure agent, an anti-oxidant agent, or a viscosity modifier.

10. A magnetorheological fluid comprising the polyol composition of claim 3, wherein the magnetorheological fluid further comprises an organomolybdenum compound, or an organothiophosphorous compound, or a combination thereof.

11. A drilling fluid comprising the polyol composition of claim 1.

12. A drilling fluid comprising the polyol composition of claim 3.

13. A lubricating fluid comprising the polyol composition of claim 1.

14. A lubrication fluid comprising the polyol composition of claim 3.

15. An adhesive comprising the polyol composition of claim 1.

16. A coating comprising the polyol composition of claim 1.

17. A grease composition comprising the polyol composition of claim 1.

18. The polyol composition of claim 1:
wherein said mono-ol comprises a compound having the formula 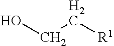
wherein R¹ comprises a residue from the oligomerization of propylene or isobutylene;
wherein said diol comprises a compound having the formula 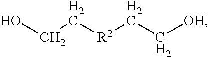
wherein said R² comprises a compound comprising from 1 to 38 carbon atoms comprising a linear alkylene, branched alkylene, cyclic alkylene, or heterocyclic alkylene, or any combination thereof.

19. The polyol composition of claim 1, wherein said mono-ol comprises (2-hydroxyethyl)cyclohexane, N-(6-hydroxyhexyl)piperidine, 3-phenylpropanol, 4-phenylbutanol, 4-(3-hydroxypropyl-pyridine, 3-(4-hydroxybutyl)furan, and any combination thereof.

20. The polyol composition of claim 1 wherein said polyol comprises a compound having the formula 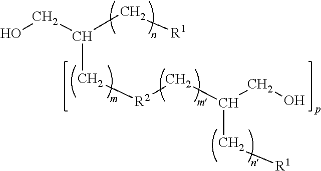
wherein each R¹, independently, is a compound having the formula R—CH₂—CH₂— wherein R comprises from about 1 to about 40 carbon atoms and is a linear alkyl, branched alkyl, cyclic alkyl, or heterocyclic alkyl, or any combination thereof; or
a compound having the formula Ar′(-Q-CH₂—CH₂)— where Q is —(CR′₂)ⱼ—, wherein j is from 1 to about 10, each R′, independently, is hydrogen or R¹ as defined hereinabove, and wherein Ar has from 4 to about 37 carbon atoms or is phenyl, pyridyl, furanyl, o-, m-, p-alkylphenyl, o-, m-, or p-phenoxyphenyl, or any other ortho, meta, para substituents compatible with the reaction conditions of the mono-ol with said diol; and
wherein said R² comprises a compound having from about 1 to about 38 carbon atoms and is a linear alkylene, branched alkylene, cyclic alkylene, or heterocyclic alkylene, or any combination thereof; or
a compound having the formula Ar(CH₂—)₂ wherein Ar comprises from about 4 to about 36 carbon atoms and is m-phenyl, m- or p- or p′-diphenyl ether, or m- or m′ or p- or p′-diphenylmethane, or any other ortho, meta, or para substituents compatible with the reaction conditions of said mono-ol with said diol; or
a compound having the formula Ar′(-Q-(CH₂)ₖ—CH₂)₂ where each Q, independently, is —(CR″₂)ₙ—, where each n, independently, is 0 or 1 to about 10 and each R″, independently, can be hydrogen or R² as defined hereinabove, where each k, independently, is 0 or 1, wherein Ar′ is from about 4 to about 32 carbon atoms, and wherein Ar′ can be a, o-, m-, or p-alkylphenyl, o-, m-, or p-phenoxyphenyl, or any other ortho, meta, or para substituents compatible with the reaction conditions of said mono-ol with said diol; and
wherein said R, Ar, and Ar′ of said diol can, independently, be the same or different than said R, Ar, and Ar′ of said mono-ol; and
wherein n is 0 when m is 2, or n is 2 when m is 0,
n′ is 0 when m′ is 2, or n′ is 2 when m′ is 0;
for diol residues not adjacent to terminating mono-ols,
m is 0 when m′ is 2 (of adjacent repeat units)
m is 2 when m′ is 0 (of adjacent repeat units); and
p is 4 on average two times the molar ratio of diol to mono-ol and need not be integral.

21. The polyol composition of claim 1, wherein said polyol comprises a compound having the formula 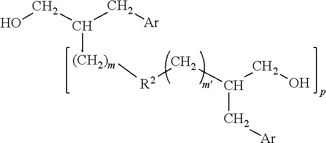
wherein each Ar, independently, comprises from about 4 to about 41 carbon atoms, or is phenyl, pyridyl, furanyl, m- or p alkylphenyl, m- or p-phenolxyphenol, or any other meta or para substituents compatible with the reaction conditions;
wherein said R² comprises a compound having from about 1 to about 38 carbon atoms comprising a linear alkylene, branched alkylene, cyclic alkylene, or heterocyclic alkylene, or any combination thereof; or
a compound having the formula Ar(CH₂—)₂— wherein Ar comprises from about 4 to about 36 carbon atoms and is m-phenyl, m- or m′- or p- or p′-diphenyl ether, or m- or m′ or p- or p′-diphenylmethane, or any other ortho, meta, or para substituents compatible with the reaction conditions of said mono-ol with said diol; or
a compound having the formula Ar(-Q-(CH₂)ₖ—CH₂)₂— where each Q, independently, is —(CR″₂)ₙ—, where each n, independently, is 0 or 1 to about 10 and each R″, independently, can be hydrogen or R² as defined hereinabove, where each k, independently, is 0 or 1, wherein Ar′ is from about 4 to about 32 carbon atoms, and wherein Ar can be a, o-, m-, or p-alkylphenyl, o-, m-, or p-phenoxyphenyl, or any other ortho, meta, or para substituents compatible with the reaction conditions of said mono-ol with said diol; and
where m=0 and m′═O when adjacent to a terminal mono-ol
and for diol residues not adjacent to terminating mono-ols
m=0 when m′=2 (of adjacent repeat units)
or m=2 when m′═O (of adjacent repeat units)
and the average value of all p's equals two times the mole ratio diol to mon-ol.

22. An engine lubricating fluid comprising the polyol composition of claim 20, wherein the mole ratio of said one or more diols to said one or more mono-ols is from about 0.5 to about 2.0.

23. The polyol composition of claim 22, wherein said mono-ol is a branched mono-ol.

24. The polyol composition of claim 22, including zinc dialkyldithiophosphate.

25. The polyol composition of claim 24, including a base oil comprising natural fatty oils, mineral oils, polyphenylethers, dibasic acid esters, neopentylpolyol esters, phosphate esters, synthetic cycloparaffins and synthetic paraffins, synthetic unsaturated hydrocarbon oils, monobasic acid esters, glycol esters and ethers, silicate esters, silicone oils, silicone copolymers, synthetic hydrocarbons, poly-alpha-olefins derived from oligomerizing terminal alkenes such as 1-butene, 1-hexene, and the like, poly-alkylene-glycols such as oligomeric poly(propylene oxide), poly(butylenes oxide), and various alkylene oxide copolymers, naphthenic oils, diesel oils, and mixtures or blends thereof.

26. A friction reducing additive comprising the polyol composition of claim 20.

27. A drilling fluid additive comprising the polyol composition of claim 20, and wherein the mole ratio of said one or more diols to said one or more mono-ols is from about 1.5 to about 5.0.
